(19) United States
(12) Patent Application Publication
(10) Pub. No.: US 2026/0193333 A1
(43) Pub. Date: Jul. 9, 2026

(54) METHODS OF INHIBITING FIBRIL GROWTH

(71) Applicant: University of Tennessee Research Foundation, Knoxville, TN (US)

(72) Inventors: Jonathan S. WALL, Knoxville, TN (US); Angela Dawn WILLIAMS, Clinton, TN (US)

(21) Appl. No.: 19/511,500

(22) PCT Filed: Aug. 15, 2024
(86) PCT No.: PCT/US2024/042578

§ 371 Date: Feb. 13, 2026

Related U.S. Application Data
(60) Provisional application No. 63/520,007, filed on Aug. 16, 2023.

(51) Int. Cl. C07K 16/18; A61K 38/00; A61K 47/68; A61P 43/00; C07K 14/47

(52) U.S. Cl. CPC C07K 16/18; A61K 47/6811; A61K 47/6889; A61P 43/00; C07K 14/4711; A61K 38/00; C07K 2317/24; C07K 2317/52; C07K 2317/565; C07K 2317/92; C07K 2319/00

(57) ABSTRACT

The present disclosure relates in some aspects to methods and compositions for reducing or inhibiting amyloid fibril growth.

### CROSS-REFERENCE TO RELATED APPLICATIONS

This application claims priority to U.S. Provisional Application No. 63/520,007, filed on Aug. 16, 2023, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165992001340seqlist.xml, date recorded: Aug. 2, 2024, size: 82 KB).

### FIELD

The present disclosure relates in some aspects to methods of preventing or reducing amyloid fibril growth.

### BACKGROUND

Amyloidosis is a fatal protein-folding disorder characterized by the aggregation and deposition of proteinaceous fibrils and heparan sulfate proteoglycan in vital organs and tissues, including the kidneys, pancreas, liver, spleen, nervous tissue and heart. The accumulation of amyloid leads to organ dysfunction and severe morbidity or death. Transthyretin-associated (ATTR) amyloidosis and light chain-associated (AL) amyloidosis are the two most common systemic amyloidosis diseases, wherein the median survival is less than 2 years. Patients diagnosed with severe cardiac AL amyloidosis have a median survival that is less than 5 months. This is due to rapid organ destruction, the inability to diagnose the disease before organ failure occurs, and the lack of effective anti-amyloid therapeutics.

To date, no treatments for clearing amyloid are available in the clinic; however, numerous amyloid-reactive antibodies, including anselamimab (CAEL101), birtamimab (NEOD001), dezamizumab (GSK2398852), solanezumab, PRX004, NI006, bapineuzumab, and aducanumab are being evaluated in clinical trials. Each of these therapeutics have limitations and/or did not meet primary outcomes in late stage clinical trials (Phase 2/3). Further, these therapeutics are specific to a single type of amyloid (e.g., AL or ATTR) and afford limited efficacy for patients suffering from the many other types of amyloidosis. Accordingly, there is still an urgent need for effective and clinically approved therapeutics that can address amyloidosis and amyloid related diseases.

### SUMMARY OF THE INVENTION

In some aspects, provided herein is a method of reducing or preventing fibril growth in an individual at risk of developing an amyloid related disease, comprising administering to the individual a therapeutically effective amount of an antibody-peptide fusion protein, wherein the antibody-peptide fusion protein comprises: (i) an amyloid-reactive peptide; and (ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

In other aspects, provided herein is a method of reducing or slowing the progression of an amyloid related disease in an individual, wherein the individual has been diagnosed with an amyloid disease, comprising administering to the individual a therapeutically effective amount of an antibody-peptide fusion protein, wherein the antibody-peptide fusion protein comprises: (i) an amyloid-reactive peptide; and (ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

In some embodiments, the amyloid fibrils comprise an amyloidogenic λ6 variable domain protein (Vλ6Wil) or an amyloidogenic immunoglobulin light chain (AL), Aβ(1-40) amyloid-like fibril or an amyloidogenic Aβ precursor protein, or serum amyloid protein A (AA).

In some embodiments, the amyloid fibrils comprise amyloidogenic forms of immunoglobulin heavy chain (AH), β2-microglobulin (Aβ2M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin (ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALβ4, or ALβL.

In some embodiments, the fibril growth is measured via an in vitro rVλ6WIL (WIL) fibril extension assay.

In some embodiments, the antibody-peptide fusion protein reduces rVλ6WIL fibril growth by at least about 20%. In some embodiments, the antibody-peptide fusion protein reduces rVλ6WIL fibril growth by about 33%. In some embodiments, the antibody-peptide fusion protein reduces the growth rate of rVλ6WIL fibril by at least about 20%. In some embodiments, the antibody-peptide fusion protein reduces the growth rate of rVλ6WIL fibril by about 33%. In some embodiments, the antibody-peptide fusion protein reduces the total amount of rVλ6WIL fibril by at least about 20%. In some embodiments, the antibody-peptide fusion protein reduces the total amount of rVλ6WIL fibril by about 33%.

In some embodiments, the amyloid related disease is systemic or localized amyloidosis. In some embodiments, the amyloid related disease is selected from the group consisting of AL, AH, Aβ2M, ATTRv, AATRwt, AA, AApoAI, AApoAII, AGel, ALys, ALECT2, AFib, ACys, ACal, AMed, AIAPP, APro, AIns, APrP, or AP amyloidosis.

In some embodiments, the individual has a genetic predisposition to an amyloid related disease. In some embodiments, the individual has a family history of an amyloid related disease. In some embodiments, the individual has an early stage of an amyloid related disease. In some embodiments, the individual has an early stage of AL amyloidosis. In some embodiments, the early stage of AL amyloidosis is diagnosed according to the Mayo Clinic system. In some embodiments, the early stage of AL amyloidosis is stage 1 AL amyloidosis.

In some embodiments, the individual has an early stage of ATTR amyloidosis. In some embodiments, the early stage of ATTR amyloidosis comprises stage 1 ATTR amyloidosis.

In some embodiments, the light chain of the antibody comprises a light chain constant region, and the heavy chain of the antibody comprises a heavy chain constant region.

In some embodiments, the spacer is selected from the group consisting of SEQ ID NOs: 23-24, 27, and 83-86. In some embodiments, the spacer is selected from the group consisting of SEQ ID NO:83 and SEQ ID NO:86.

In some embodiments, the amyloid-reactive peptide comprises an amino acid sequence selected from the group consisting of SEQ ID NOs: 1-13 comprising 0, 1, 2, 3, or 4 amino acid substitutions, insertions, or deletions. In some embodiments, the amyloid-reactive peptide comprises an amino acid sequence selected from the group consisting of SEQ ID NOs: 1-13.

In some embodiments, the antibody-peptide fusion protein comprises two heavy chains and two light chains, and wherein each light chain is linked at the C-terminal end to the amyloid-reactive peptide. In some embodiments, the antibody-peptide fusion protein comprises two heavy chains and two light chains, and wherein each light chain is linked at the C-terminal end of the light chain to the amyloid-reactive peptide.

In some embodiments, the antibody is a chimeric antibody or humanized antibody.

In some embodiments, the light chain of the antibody comprises a light chain variable domain (VL) comprising a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22, and the heavy chain of the antibody comprises a heavy chain variable domain (VH) comprising a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19.

In some embodiments, the VL comprises an amino acid sequence set forth in SEQ ID NO:36, and the VH comprises an amino acid sequence set forth in SEQ ID NO:55.

In some embodiments, the antibody is a full-length antibody comprising an Fc region. In some embodiments, the Fc region is of an IgG1 isotype.

In some embodiments, the antibody-peptide fusion protein comprises: (i) an amyloid-reactive peptide comprising the amino acid sequence set forth in SEQ ID NO:1 or SEQ ID NO:2; and (ii) an antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22; wherein the amyloid-reactive peptide and antibody are linked at the C-terminal end of the light chain, and wherein the amyloid-reactive peptide is linked to the antibody via a spacer comprising an amino acid sequence selected from the group consisting of SEQ ID NOs: 23-24, 27, 83-86.

In some embodiments, the antibody-peptide fusion protein comprises: (i) an amyloid-reactive peptide comprising the amino acid sequence set forth in SEQ ID NO:2; and (ii) an antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22; wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83.

In some embodiments, the antibody-peptide fusion protein comprises: (i) an amyloid-reactive peptide comprising the amino acid sequence set forth in SEQ ID NO:2; and (ii) an antibody that binds to a human amyloid fibrils, wherein the antibody comprises a heavy chain and a light chain, wherein the heavy chain of the antibody comprises a heavy chain variable region (VH) comprising a CDR-H1, CDR-H2, and CDR-H3 of a VH comprising the amino acid sequence set forth in SEQ ID NO:55, and the light chain of the antibody comprises a light chain variable region (VL) comprising a CDR-L1, CDR-L2, and CDR-L3 of a VL comprising the amino acid sequence set forth in SEQ ID NO:36; wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83.

In some embodiments, the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide each comprises the amino acid set forth in SEQ ID NO:89, and the third and fourth polypeptide each comprises the amino acid sequence set forth in SEQ ID NO:91

In some embodiments, the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide each comprises the amino acid set forth in SEQ ID NO:89, and the third and fourth polypeptide each comprises the amino acid sequence set forth in SEQ ID NO:93.

In some embodiments, the individual is a human.

### BRIEF DESCRIPTION OF THE DRAWINGS

The drawings illustrate certain features and advantages of this disclosure. These embodiments are not intended to limit the scope of the appended claims in any manner.

FIG. 1A depicts an exemplary amyloid reactive peptide. FIG. 1B depicts an exemplary amyloid binding antibody-peptide fusion. FIG. 1C depicts an exemplary amyloid binding antibody.

FIGS. 2A-2B depict results from a rVλ6WIL fibril extension assay with biotinylated rVλ6WIL monomer mixed with an amyloid binding peptide, p5R (SEQ ID NO: 2). FIG. 2A depicts the quantification of bound rVλ6WIL (fmoles europium) for a positive control sample containing only rVλ6WIL fibrils and biotinylated-rVλ6WIL monomer and experimental wells containing fibrils, monomer, and various concentrations of the amyloid binding peptide (2, 0.5, 0.05, 0.01, 0.005 and 0.001 μM). FIG. 2B depicts the percentage change of rVλ6WIL fibril extension compared to the control sample for each concentration of amyloid binding peptide tested in FIG. 2A.

FIGS. 3A-3B depict results from a rVλ6WIL fibril extension assay with biotinylated rVλ6WIL monomer mixed with rVλ6WIL fibrils in the presence or absence of an amyloid binding antibody-peptide fusion protein. FIG. 3A depicts the quantification of bound rVλ6WIL (fmoles europium) for a positive control sample containing no amyloid binding antibody-peptide fusion protein and experimental wells containing various concentrations of the amyloid binding antibody-peptide fusion protein (2, 0.5, 0.05, 0.01, 0.005 and 0.001 μM). FIG. 3B depicts the percentage change of rVλ6WIL fibril extension compared to the control sample.

FIGS. 4A-4B depict results from a rVλ6WIL fibril extension assay with biotinylated rVλ6WIL monomer mixed with rVλ6WIL fibrils in the presence or absence of an amyloid binding antibody. FIG. 4A depicts the quantification of bound rVλ6WIL (fmoles europium) for a positive control sample containing no amyloid binding antibody and experimental wells containing various concentrations of the amyloid binding antibody (2, 0.5, 0.05, 0.01, 0.005 and 0.001 μM). FIG. 4B depicts the percentage change of rVλ6WIL fibril extension compared to the control sample.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods of reducing or preventing fibril growth in an individual at risk of developing an amyloid related disease comprising administering to the individual an antibody-peptide fusion protein, wherein the antibody-peptide fusion protein comprises an amyloid-reactive peptide, and an antibody that binds to amyloid fibrils. In some embodiments, the antibody comprises a heavy chain and a light chain, and the amyloid-reactive peptide and the antibody are linked, for example, at the C-terminal end of the light chain, and where the amyloid-reactive peptide is linked to the antibody via a spacer.

In some embodiments, provided herein is method of reducing or preventing fibril growth in an individual at risk of developing an amyloid related disease, comprising administering to the individual a therapeutically effective amount of an antibody-peptide fusion protein, wherein the antibody-peptide fusion protein comprises: (i) an amyloid-reactive peptide; and (ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

In some embodiments, provided herein is a method of reducing or slowing the progression of an amyloid related disease in an individual, wherein the individual has been diagnosed with an amyloid disease, comprising administering to the individual a therapeutically effective amount of an antibody-peptide fusion protein, wherein the antibody-peptide fusion protein comprises: (i) an amyloid-reactive peptide; and (ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

### II. Definitions

As used herein, the singular forms “a,” “an,” and “the,” refer to both the singular as well as plural, unless the context clearly indicates otherwise. The abbreviation, “e.g.” is derived from the Latin exempli gratia, and is used herein to indicate a non-limiting example. Thus, the abbreviation “e.g.” is synonymous with the term “for example.” As used herein, the term “comprises” means “includes.”

Ranges can be expressed herein as from “about” one particular value, and/or to “about” another particular value. When such a range is expressed, another aspect includes from the one particular value of the range and/or to the other particular value of the range. It will be further understood that the endpoints of each of the ranges are significant both in relation to the other endpoint, and independently of the other endpoint. Similarly, when values are expressed as approximations, by use of the antecedent “about,” it will be understood that the particular value forms another aspect. In certain example embodiments, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about. Further, terms used herein such as “example,” “exemplary,” or “exemplified,” are not meant to show preference, but rather to explain that the aspect discussed thereafter is merely one example of the aspect presented.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. In case of conflict, the present specification, including explanations of terms, will control. In addition, the materials, methods, and examples are illustrative only and not intended to be limiting.

To facilitate review of the various embodiments of this disclosure, the following explanations of specific terms are provided:

The terms “amyloids,” “amyloid deposits,” “amyloid fibrils,” and “amyloid fibers” refer to insoluble fibrous protein aggregates sharing specific structural traits. The protein aggregates have a tertiary structure, for example, that is formed by aggregation of any of several different proteins and that consists of an ordered arrangement of p sheets stacked perpendicular to a fiber axis. See Sunde et al., J. Mol. Biol. (1997) 273:729-39. Abnormal accumulation of amyloid in organs may lead to amyloidosis. Although they are diverse in their occurrence, all amyloids have common morphologic properties in that they stain with specific dyes such as Congo red and have a characteristic red-green birefringent appearance in polarized light after staining. Amyloids also share common ultrastructural features and common x-ray diffraction and infrared spectra.

The term “amyloidosis” refers to a pathological condition or disease characterized by the presence of amyloid, such as the presence of amyloid deposits. “Amyloid diseases” or “amyloidosis” are diseases associated with the formation, deposition, accumulation or persistence of amyloid fibrils. Such diseases include, but are not limited to, AA amyloidosis, AL amyloidosis, ATTR amyloidosis, ALECT2 amyloidosis, and AIAPP amyloidosis of type II diabetes.

The term “amyloidogenic” refers to producing or tendency to produce amyloid deposits. For example, certain soluble monomeric proteins can undergo extensive conformational changes leading to their aggregation into well-ordered, unbranching, 8- to 10-nm wide fibrils, which culminate in the formation of amyloid aggregates. More than thirty proteins, for example, have been found to form amyloid deposits (or amyloids) in man. Not all proteins within the class of diverse proteins, such as immunoglobulin light chains, are capable of forming amyloid, i.e., some immunoglobulin light chains are non-amyloidogenic, meaning that they do not tend to form amyloids. Other proteins of the class, however, can form amyloid deposits and are thus amyloidogenic. Furthermore, within the class of light chain proteins, some may be deemed more “amyloidogenic” than others based upon the ease with which they form amyloid fibrils. Certain light chain proteins are deemed non-amyloidogenic or less amyloidogenic because of their inability to readily form amyloid fibrils in patients or in vitro.

The terms “clear” or “clearance” refer to reducing or removing by a measurable degree. For example, the clearance of an amyloid deposit as described herein relates to reducing or removing the deposit to a measurable or discernable degree. Clearance may result in 100% removal, but is not required to. Rather, clearance may result in less than 100% removal, such as about 10%, 20%, 30%, 40%, 50%, 60% or more removal.

As used herein, the term “conjugate” refers to the product of coupling or joining of two or more materials, the resulting product having at least two distinct elements, such as at least two domains. The coupled materials may be the same or may be different. Such a coupling may be via one or more linking groups. A “protein conjugate,” for example, results from the coupling of two or more amino acid sequences. A conjugate of two proteins, for example, results in a single protein that has a domain corresponding to each of the individually joined proteins.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains,

The term “constant region” refers to the portion of an immunoglobulin molecule having a more conserved amino acid sequence relative to the other portion of the immunoglobulin, the variable domain, which contains the antigen binding site. The constant region contains the CR1, CR2 and CR3 domains (also termed CH1, CH2, and CH3; collectively, CH) of the heavy chain and the CHL (or CL or CL1) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md, (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“u”) and lambda (“X”), based on the amino acid sequences of their constant domains,

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody-fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association, In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment contains the heavy- and light-chain variable domains and also contains the constant domain of the light chain and the first constant domain (CHI.) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckfhun, in The Pharmacology of Monoclonal Antibodies, vol. 113, Rosenburg and Moore eds., (Springer-Verlag, New York, 1994), pp. 269-315.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal.” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different, determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The term “amyloid reactive antibody” refers to an antibody that binds to an amyloid. As used herein the term amyloid reactive antibody and antibody that binds to amyloid fibrils are used interchangeably.

The term “amyloid reactive peptide” refers to a peptide that binds to any of various amyloid fibrils such as, but not limited to, amyloidogenic λ6 variable domain protein (Vλ6Wil) or an amyloidogenic immunoglobulin light chain (AL), A13(1-40) amyloid-like fibril or an amyloidogenic AP precursor protein, or serum amyloid protein A (AA). In other embodiments, the amyloids bound by the antibody-peptide fusion protein comprise amyloidogenic forms of immunoglobulin heavy chain (AH), β2-microglobulin (Aβ2M), transthyretin variants (ATTR), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin ((ACal), lactadherin (AMed), islet amyloid polypeptide (AIAPP), prolactin (APro), insulin (Ahs), prior protein (APrP); a-synuclein (AαSyn), tau (ATau), atrial natriuretic factor (AANF), or IAAP, ALκ4, A1λ1 other amyloidogenic peptides.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al, Hybridoma, 1.4 (3): 253-260 (0.1995), Harlow et al., Antibodies: A Laboratory Manual, (Cold Spring Harbor Laboratory Press, 2nd ed. 1988); Hammerling et al, in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al, Nature, 352: 624-628 (1991); Marks et al, J. Mol. Biol. 222: 581-597 (1992); Sidhu et al, J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits 11 et al, Proc. Natl. Acad. Sci. USA 90: 2551 (1993); Jakobovits et al, Nature 362: 255-258 (1993); Bruggemann et al, Year in Immunol 7:33 (1993); U.S. Pat. Nos. 5,545,807; 5,545,806; 5,569,825; 5,625,126; 5,633,425; and U.S. Pat. No. 5,661,016; Marks et al, Bio/Technology 10: 779-783 (1992); Lonberg et al, Nature 368: 856-859 (1994); Morrison, Nature 368: 812-813 (1994); Fishwild et al, Nature Biotechnol. 14: 845-851 (1996); Neuberger, Nature Biotechnol 14: 826 (1996); and Lonberg and Huszar, Intern. Rev. Immunol 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al, Proc. Natl Acad. Sci. USA 81:6851-6855 (1984)). Chimeric antibodies include primatized antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a CDR of the recipient are replaced by residues from a CDR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al, Nature 321:522-525 (1986); Riechmann et al, Nature 332:323-329 (1988); and Presta, Curr. Op. Struct. Biol 2:593-596 (1992), See also, e.g., Vaswani and Hamilton, Ann. Allergy, Asthma & Immunol. 1: 105-115 (1998); Harris, Biochem. Soc. Transactions 23: 1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al, J. Mol. Biol., 222:581 (1991). Also available for the preparation of human monoclonal antibodies are methods described in Cole et al, Monoclonal Antibodies and Cancer Therapy, Alan R. Liss, p. 77 (1985); Boerner et al, J. Immunol., 147(1):86-95 (1991). See also van Dijk and van de Winkel, Curr. Opin. Pharmacol., 5: 368-74 (2001), Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al, Proc. Natl. Acad. Sci. USA, 103:3557-3562 (2006) regarding human antibodies generated via a human B-cell hybridoma technology.

The term “complementarity-determining region” or “CDR,” when used herein refers to the regions of an antibody-variable domain that bind to an epitope, such as human amyloid fibrils. Generally, antibodies comprise six CDRs; three in the VH (H1, H2, H3), and three in the VL (L1, L2, L3). In native antibodies, H3 and L3 display the most diversity of the six CDRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo ed., Human Press, Totowa, NJ, 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of CDR delineations are in use and are encompassed herein. In some embodiments, the CDRs may be Kabat CDRs, which are based on sequence variability and are the most commonly used (Kabat et al., supra). In some embodiments, the CDRs may be Chothia CDRs. Chothia refers instead to the location of the structural loops (Chothia and Lesk J. Mol. Biol. 196:901-917 (1987)). In some embodiments, the CDRs may be AbM CDRs. The AbM CDRs represent a compromise between the Kabat CDRs and Chothia structural loops, and are used by Oxford Molecular's AbM antibody-modeling software. In some embodiments, the CDRs may be “contact” CDRs. The “contact” CDRs are based on an analysis of the available complex crystal structures. The residues from each of these CDRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

CDRs may comprise “extended CDRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-CDR definitions.

“Framework” or “FR” residues are those variable domain residues other than the CDR residues as herein defined.

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 M, <100 μM, <10 nM, <1 nM, or <0.1 nM. In certain embodiments, an antibody specifically binds to an epitope on a protein that is conserved among the protein from different species. In another embodiment, specific binding can include, but does not require exclusive binding.

The term “effective amount” or “therapeutically effective amount as used herein refer to the amount of agent that is sufficient to prevent, treat (including prophylaxis), reduce and/or ameliorate the symptoms and/or underlying causes of any of a disorder or disease, for example to prevent, inhibit, and/or amyloidosis. In some embodiments, an “effective amount” is sufficient to reduce or eliminate a symptom of a disease. An effective amount can be administered one or more times. For example, an effective amount of a peptide is an amount that is sufficient to bind an amyloid. A peptide may be effective, for example, when parenterally administered in amounts above about 1 μg per kg of body weight to about 30 mg/kg.

As used herein, the term “inhibit” is to reduce by a measurable degree. Inhibition does not, for example, require complete loss of function or complete cessation of the aspect being measured. For example, inhibiting fibril formation can mean stopping further growth of the fibril, slowing further growth of the fibril, or reducing the size of the fibril.

With regard to amyloid fibril formation, “reducing fibril growth” refers to the reduction in the rate of fibril formation or a reduction in the total amount of amyloid fibrils. For example, reducing fibril growth may result in a reduction of about 10%, 20%, 30%, 40%, 50%, 60% or more of an amyloid deposit as compared to a control. Moreover, reducing fibril growth may result in a reduction in the rate of fibril formation of about 10%, 20%, 30%, 40%, 50%, 60% or more as compared to a control. As used herein, “preventing fibril growth” refers to preventing further fibril growth after administration of the antibody-peptide fusion protein disclosed herein.

The term “label” refers to any detectable compound or composition that is conjugated directly or indirectly to another molecule to facilitate detection of that molecule. Specific, non-limiting examples of labels include fluorescent tags, chemiluminescent tags, haptens, enzymatic linkages, and radioactive isotopes. A protein that is “detectably-labeled,” for example, means that the presence of the protein can be determined by a label associated with the protein.

An “isolated” biological component, such as a peptide (for example one or more of the peptides disclosed herein), cell, nucleic acid, or serum samples has been substantially separated, produced apart from, or purified away from other biological components in the cell of the organism in which the component naturally occurs, for instance, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a cell as well as chemically synthesized peptide and nucleic acids. The term “isolated” or “purified” does not require absolute purity; rather, it is intended as a relative term. Thus, for example, an isolated peptide preparation is one in which the peptide or protein is more enriched than the peptide or protein is in its natural environment within a cell. Preferably, a preparation is purified such that the protein or peptide represents at least 50% of the total peptide or protein content of the preparation, such as at least 50%, at least 60%, at least 70%, at least 80%, at least 90%, at least 95%, or even at least 99% of the peptide or protein concentration.

As used herein, the term “join,” “joined,” “link,” or “linked” refers to any method known in the art for functionally connecting proteins and/or protein domains. For example, one protein domain may be linked to another protein domain via a covalent bond, such as in an antibody-peptide fusion protein, with or without intervening sequences or domains. Joined also includes, for example, the integration of two sequences together, such as placing two nucleic acid sequences together in the same nucleic acid strand so that the sequences are expressed together.

The term “nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

The term “cDNA” refers to a DNA that is complementary or identical to an mRNA, in either single stranded or double stranded form.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “pharmaceutically acceptable carriers” refers to pharmaceutically acceptable carriers of use that are conventional. Remington's Pharmaceutical Sciences, by E. W. Martin, Mack Publishing Co., Easton, PA, 19^{th} Edition (1995), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The term “polypeptide” refers to a polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “purified” does not require absolute purity; rather, it is intended as a relative term. Thus, for example, a purified protein preparation is one in which the protein referred to is more pure than the protein in its natural environment within a cell or within a production reaction chamber (as appropriate).

With regard to a recombinant nucleic acid, the term “recombinant” refers to a recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

The term “sequence identity” refers to the similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene 73: 237-244, 1988; Higgins & Sharp CABIOS 5: 151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

As used herein, a first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

The term “vector” refers to a nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses. The term vector includes plasmids, linear nucleic acid molecules, and as described throughout adenovirus vectors and adenoviruses.

A “subject” or an “individual” refers to a mammal, for example, a human. The subject may be a human patient A subject may be a patient suffering from or suspected of suffering from an early stage of an amyloid related disease or condition and may be in need of treatment or diagnosis or may be in need of monitoring for the progression of the disease or condition. The patient may also be receiving a treatment therapy that needs to be monitored for efficacy. In some example embodiments, a subject includes an individual suffering from systemic amyloidosis.

Preferably, residue positions which are not identical differ by conservative amino acid substitutions. The term “conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. (Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of LI, 50-55 of L2, 89-96 of L3, 31-35B of HI, 50-58 of H2, and 95-102 of H3. (See Almagro and Fransson, Front. Biosci. 13: 1619-1633 (2008).)

### III. Methods of Treatment

Provided herein are methods of reducing or preventing fibril growth in an individual at risk of developing an amyloid related disease, comprising administering to the individual a therapeutically effective amount of an antibody-peptide fusion protein. Alternatively, in some embodiments, provided herein are methods of reducing or slowing the progression of an amyloid related disease in an individual, wherein the individual has been diagnosed with an amyloid disease, comprising administering to the individual a therapeutically effective amount of an antibody-peptide fusion protein. In some embodiments, the antibody-peptide fusion protein comprises: (i) an amyloid-reactive peptide; and (ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

Also provided herein are methods of reducing or preventing fibril growth in an individual having an amyloid related disease, comprising administering to the individual a therapeutically effective amount of an antibody-peptide fusion protein. Also provided herein are methods of reducing or preventing fibril growth in an individual in whom amyloid fibrils have been identified, comprising administering to the individual a therapeutically effective amount of an antibody-peptide fusion protein. Also provided herein are methods of reducing or preventing fibril growth in an individual in whom amyloid fibrils have been identified, comprising administering to the individual a therapeutically effective amount of an antibody-peptide fusion protein. Also provided herein are methods of reducing or preventing fibril growth in an individual, wherein the individual has been diagnosed with an amyloid disease, comprising administering to the individual a therapeutically effective amount of an antibody-peptide fusion protein.

In some embodiments, the antibody-peptide fusion protein reduces or prevents amyloid fibril growth in vitro and/or in vivo. In some embodiments, an in vitro rVλ6WIL fibril extension assay is utilized to measure the effect of the antibody-peptide fusion protein on amyloid fibril growth. In some embodiments, the rVλ6WIL fibril extension assay measures rVλ6WIL fibril extension in the presence of the antibody-peptide fusion protein described herein. In some embodiments, the antibody-peptide fusion protein reduces rVλ6WIL fibril extension by between 10% and 50%. In some embodiments, the antibody-peptide fusion protein reduces rVλ6WIL fibril extension by about 10%, 15%, 20%, 25%, 30%, 35%, 40%, 45%, or 50%. In some embodiments, the antibody-peptide fusion protein reduces rVλ6WIL fibril extension by about 33%.

In some embodiments, the methods of reducing or preventing fibril growth in an individual at risk of developing an amyloid related disease comprise administering to the individual a therapeutically effective amount of an amyloid-reactive antibody. Alternatively, in some embodiments, the methods of reducing or slowing the progression of an amyloid related disease in an individual, wherein the individual has been diagnosed with an amyloid disease, comprise administering to the individual a therapeutically effective amount of an amyloid-reactive antibody. In some embodiments, the amyloid-reactive antibody binds to amyloid fibrils, and wherein the antibody comprises a heavy chain and a light chain.

In some embodiments, the amyloid-reactive antibody reduces or prevents amyloid fibril growth in vitro and/or in vivo. In some embodiments, the amyloid-reactive antibody reduces rVλ6WIL fibril extension by between 10% and 95%. In some embodiments, amyloid-reactive antibody reduces rVλ6WIL fibril extension by about 10%, 20%, 30%, 40%, 50%, 60%, 70%, 80%, 85%, 90%, 95% or 99%. In some embodiments, the amyloid-reactive antibody reduces rVλ6WIL fibril extension by about 89%.

In some embodiments, fibril growth can occur through the recruitment of fibril monomers at either the fibril ends or along the long axis. In some embodiments, the antibody-peptide fusion protein blocks amyloid fibril growth by sequestering fibril monomers or misfolded fibril monomers. In some embodiments, the antibody-peptide fusion protein blocks amyloid fibril growth by blocking access to extension sites at either end of the amyloid fibril. In some embodiments, the antibody-peptide fusion protein blocks amyloid fibril growth by blocking access to extension sites along the long axis of the amyloid fibril. In some embodiments, the antibody-peptide fusion protein blocks amyloid fibril growth through a combination of these mechanisms.

In some embodiments, the individual is at risk of developing an amyloid related disease. In some embodiments, the individual has a family history of an amyloid disease. In some embodiments, the individual has a genetic predisposition to an amyloid disease. In some embodiments, the individual has a genetic predisposition to AA amyloidosis. In some embodiments, the genetic predisposition to AA amyloidosis comprises mutations within the amyloidogenic precursor protein SAAL. In some embodiments, the genetic predisposition to AA amyloidosis comprises polymorphisms of the mannose-binding lectin 2 (MBL-2) gene. In some embodiments, the amyloidosis is a hereditary form of amyloidosis. In some embodiments, the hereditary form of amyloidosis comprises mutated transthyretin (TTR) associated cardiac amyloidosis. In some embodiments, TTR associated cardiac amyloidosis disproportionately afflicts Black Americans. In some embodiments the mutated form of TTR comprises a point mutation resulting in an amino acid substitution e.g., of isoleucine for valine at position 122 (Val122Ile). Those having ordinary skill in the art can readily identify individuals who are susceptible to amyloid related disease for whom the methods of the invention are particularly useful.

Additionally, the present invention is particularly useful to ameliorate or prevent the progression of amyloid disease symptoms. In some embodiments, the individual has an early stage of an amyloid disease. In some embodiments, the individual has one or more symptoms of an amyloid disease. In some embodiments amyloid disease symptoms comprise swollen ankles and legs, severe fatigue, shortness of breath, significant weight loss, difficulty in swallowing, tingling, numbness or pain in the hands wrist or feet, an enlarged tongue, irregular heartbeat, diarrhea, and/or easy bruising. In some embodiments, treatment with a therapeutically effective amount of an antibody-peptide fusion protein delays the progression of an individual with a diagnosed amyloid related disease. In some embodiments, one or more of these symptoms is ameliorated and/or stabilized following treatment with the antibody-peptide fusion protein.

In some embodiments, the individual has an early stage of an amyloid related disease. In some embodiments the individual has an early stage of amyloid light-chain amyloidosis (AL). In some embodiments, the AL amyloidosis is staged according to the Mayo Clinic system (Kumar S. et al., J. Clin. Oncol. 2012; 30:989-995).

In some embodiments, the individual has an early stage of hereditary or wild-type ATTR amyloidosis. In some embodiments, the hereditary or wild-type ATTR amyloidosis is staged according to an ATTR staging system using the cardiac biomarker NT-proBNP and the kidney biomarker eGFR (Gillmore J. D. et al., Eur Heart J. 2018; 39(30):2799-2806).

In some embodiments, the amyloid deposits may contribute to the pathology of a disease. In other embodiments, the amyloid deposits may be indicative of amyloidosis or an amyloid-related disease in an individual. In some embodiments, the amyloid-reactive antibody-peptide fusion protein or amyloid-reactive antibody binds to amyloids in an individual with an amyloidosis. In some embodiments, the amyloidosis is localized to a specific tissue or organ system, such as the pancreas, liver, the heart, or the central nervous system.

In other embodiments, the amyloidosis is a systemic amyloidosis. In some embodiments, the amyloidosis is a familial amyloidosis. In other embodiments, the amyloidosis is a sporadic amyloidosis. In some embodiments, the amyloidosis or amyloid-related disease is AA amyloidosis, AL amyloidosis, AH amyloidosis, Ap amyloidosis, ATTRv amyloidosis, ATTRwt amyloidosis, ALECT2 amyloidosis, and AIAPP amyloidosis of type II diabetes, Alzheimer's disease, Down's syndrome, hereditary cerebral hemorrhage with amyloidosis of the Dutch type, cerebral beta-amyloid angiopathy, spongiform encelohalopathy, thyroid tumors, Parkinson's disease, dementia with Lewis bodies, a tauopathy, Huntington's disease, senile systemic amyloidosis, familial hemodialysis, senile systemic aging, aging pituitary disorder, iatrogenic syndrome, spongiform encephalopathies, reactive chronic inflammation, thyroid tumors, myeloma or other forms of cancer. In some embodiments, the amyloid related disease is selected from the group consisting of AL, AH, Aβ2M, ATTRv, ATTRwt, AA, AApoAI, AApoAII, AApoAIV, AApoCII, AApoCIII, AGel, ALys, ALECT2, AFib, ACys, ACal, AMed, AIAPP, APro, AIns, APrP, ASPC, AGal7, ACor, Aker, ALac, AOAPP, ASem1, AEnf, or Aβ amyloidosis. In some embodiments, treatment with the amyloid-reactive antibody-peptide fusion protein or amyloid-reactive antibody results in the clearance of amyloid. In some embodiments, the amyloid-reactive antibody-peptide fusion protein or amyloid-reactive antibody binds to amyloids associated with normal aging. In other embodiments, the amyloid-reactive antibody-peptide fusion protein or amyloid-reactive antibody is used in the diagnosis, treatment, or prognosis of an amyloidosis or amyloid-related disease in an individual.

In some embodiments, the amyloid related disease is localized amyloidosis.

In some embodiments, the amyloid-reactive antibody-peptide fusion protein or amyloid-reactive antibody is administered via an intradermal, subcutaneous, intramuscular, intracardiac, intravascular, intravenous, intra-ocular, intra-arterial, epidural, intraspinal, extracorporeal, intrathecal, intraperitoneal, intrapleural, intraluminal, intravitreal, intracavernous, intraventricular, intra-bone, intra-articular, intracellular, or pulmonary route.

In some embodiments, the individual is a mammal such as primate, bovine, rodent, or pig. In some embodiments, the individual is a human.

### IV. Antibody-Peptide Fusion Proteins

In some aspects, the methods provided herein comprise reducing or preventing amyloid fibril growth by administering an antibody-peptide fusion protein. Alternatively, in some embodiments, the methods provided herein comprise reducing or slowing the progression of an amyloid related disease in an individual by administering an antibody-peptide fusion protein. Such antibody-peptide fusion proteins include, for example, amyloid-reactive peptides that are linked to an antibody, such as through extension of the N-terminus or C-terminus of the light chain of the antibody or a fragment thereof or via or the N-terminus or C-terminus of the heavy chain of the antibody or a fragment thereof, thereby forming a peptide-antibody fusion. In some embodiments, the amyloid-reactive peptide is linked to the antibody via a spacer. In some embodiments, the spacer comprises an amino acid sequence selected from the group consisting of SEQ ID NOs: 23-24, 27, and 83-86. In some embodiments, the amyloid-reactive peptide is linked to the antibody via a spacer comprising an amino acid sequence selected from the group consisting of SEQ ID NOs: 83-86. The antibody-peptide fusion proteins can be used to delay or prevent amyloid fibril growth and reduce or slow the progression of an amyloid related disease in a subject having amyloidosis or suspected of having amyloidosis, for example, such as by administering the antibody-peptide fusion proteins to the subject.

In some embodiments, provided herein is an antibody-peptide fusion protein, comprising: an amyloid-reactive peptide; and an antibody that reduces or prevents amyloid fibril growth or slows the progression of an amyloid related disease. In some embodiments, the antibody comprises a heavy chain comprising a heavy chain variable region (VH) and a light chain comprising a light chain variable region (VL). In some embodiments, the amyloid-reactive peptide and the antibody are linked at the N- and/or C-terminal end of the light chain and/or the N- and/or C-terminal end of the heavy chain. In some embodiments, the antibody-peptide fusion protein comprises more than one amyloid-reactive peptide linked to the antibody. In some embodiments, the amyloid-reactive peptide is linked to the antibody via a spacer. In some embodiments, the amyloid-reactive peptide is linked to the antibody via a spacer comprising the amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86. In some embodiments, the amyloid-reactive peptide and antibody are linked at the C-terminal end of the light chain. In some embodiments, the amyloid-reactive peptide and antibody are linked at the C-terminal end of the heavy chain. In some embodiments, the antibody is a full length antibody. In some embodiment, the amyloid-reactive peptide comprises an amino acid sequence as shown in Table 1, below.

In some embodiments, the amyloid-reactive peptide antibody fusion comprises a heavy chain in N- to C-terminal direction comprising in order an amyloid reactive peptide, a spacer, a VH, a CH1, a CH2, and a CH3. In some embodiments, the amyloid-reactive peptide antibody fusion comprises a heavy chain in N- to C-terminal direction in order a VH, a CH1, a CH2, a CH3, a spacer, and an amyloid reactive peptide. In some embodiments, the amyloid-reactive peptide antibody fusion comprises a light chain in N- to C-terminal direction in order an amyloid reactive peptide, a spacer, a VL, and a CL. In some embodiments, the amyloid-reactive peptide antibody fusion comprises a light chain in N- to C-terminal direction in order a VL, and a CL, a spacer, and an amyloid reactive peptide.

TABLE 1
Exemplary Amyloid-Reactive Peptide Sequences

| Peptide | Primary sequence | SEQ ID NO |
|---|---|---|
| P5 | KAQKA QAKQA KQAQK AQKAQ AKQAK Q | SEQ ID NO: 1 |
| P5R | RAQRA QARQA RQAQR AQRAQ ARQAR Q | SEQ ID NO: 2 |
| P8 | KAKAK AKAKA KAKAK | SEQ ID NO: 3 |
| P9 | KAQAK AQAKA QAKAQ AKAQA KAQAK AQAK | SEQ ID NO: 4 |
| P19 | KAQQA QAKQA QQAQK AQQAQ AKQAQ Q | SEQ ID NO: 5 |
| P20 | QAQKA QAQQA KQAQQ AQKAQ AQQAK Q | SEQ ID NO: 6 |
| P31 | KAQKA QAKQA KQAQK AQKAQ AKQAK Q | SEQ ID NO: 7 |
| P37 | KTVKT VTKVT KVTVK TVKTV TKVTK V | SEQ ID NO: 8 |
| P42 | VYKVK TKVKT KVKTK VKT | SEQ ID NO: 9 |
| P43 | AQAYS KAQKA QAKQA KQAQK AQKAQ | SEQ ID NO: 10 |
| | AKAK Q | |
| P44 | AQAYA RAQRA QARQA RQAQR AQRAQ | SEQ ID NO: 11 |
| | ARQAR Q | |
| P5+14 | KAQKA QAKQA KQAQK AQKAQ AKQAK | SEQ ID NO: 12 |
| | QAQKA QKAQA KQAKQ | |
| P5R+14 | RAQRA QARQA RQAQR AQRAQ ARQAR | SEQ ID NO: 13 |
| | QAQRA QRAQA RQARQ | |

Without wishing to be bound by any particular theory, it is believed that the amyloid-reactive peptide of the antibody-peptide fusion protein, when administered to a subject, targets the antibody-peptide fusion protein to the amyloid deposits. The Fc domain then triggers an immune response at the site of the amyloid, thereby resulting in removal of the amyloid, such as by opsonization, and can inhibit or slow the formation of amyloid. In addition, the antibody-peptide fusion protein is believed to have a longer half-life than the amyloid-reactive peptides alone. For example, the circulating half-life of an IgG in humans is approximately 21 days whereas the half-life of the amyloid-reactive peptide alone in humans is approximatively, 11 hours. Thus, the Ig enhances the half-life of the antibody-peptide fusion protein in circulation. In some embodiments, the half-life of the antibody-peptide fusion protein is increased by about 10%, 20%, 30%, 40%, 50%, 60%, 70%, 80% or more as compared to the amyloid-reactive peptide alone. As such, the antibody-peptide fusion protein, when administered to a subject, can exert its immunostimulatory effects longer at the site of the amyloid deposit, thereby increasing the immune response at the site of the amyloid deposit. In some embodiments, the amyloid reactive peptide binds to heparan sulfate glycosaminoglycans. In some embodiments, the amyloid reactive peptide is able to bind to multiple types of amyloid fibrils. In some embodiments, the amyloid reactive peptide has pan amyloid specificity.

In some embodiments, the amyloid-reactive peptides of the antibody-peptide fusion proteins described herein comprises an amino acid sequence that is at least 80%, 85%, 90% or more identical to the amino acid sequence set forth as any one of SEQ ID NOS: 1-13, such as at least 95%, at least 96%, at least 97%, at least 98%, at least 99%, or 100% identical to the amino acid sequence set forth as any one of SEQ ID NOS: 1-13. In some embodiments, the amyloid-reactive peptides linked to the antibody or functional fragments thereof may comprise or consist of from about 10 to about 55 amino acids. The amyloid-reactive peptides of the present invention may, for example, comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. Such peptides are described, for example, in international patent application WO2016032949 and Wall et al. (PLoS One. 2013 Jun. 4; 8(6):e66181), which are hereby incorporated herein in their entirety. In some embodiments, the amyloid-reactive peptide comprises an amino acid sequence having at least 80%, 85%, 90%, 95% or more sequence identity to any one of the amino acid sequences set forth as SEQ ID NOs: 1-13. In some embodiments, the amyloid-reactive peptide comprises an amino acid sequence having at least 80%, 85%, 90%, 95% or more sequence identity to any one of the amino acid sequences set forth as SEQ ID NOs: 1-13. In some embodiments, the amyloid-reactive peptide comprises the amino acid sequences set forth as SEQ ID NOs: 1-13 comprising one or more amino acid substitutions. In some embodiments, the amyloid-reactive peptide comprises an amino acid sequence set forth in SEQ ID NO: 1. In some embodiments, the amyloid-reactive peptide comprises an amino acid sequence set forth in SEQ ID NO: 2. In some embodiments, the amyloid-reactive peptide comprises an amino acid sequence set forth in SEQ ID NO:12. In some embodiments, the amyloid-reactive peptide comprises an amino acid sequence set forth in SEQ ID NO:13. In some embodiments, the amyloid-reactive peptide comprises the amino acid sequence set forth in any one or SEQ ID NOs:1-13.

TABLE 2
Amino acid sequences of m11-1F4 CDRs

| 11-1F4 | Amino Acid | SEQ ID |
|---|---|---|
| CDR | Sequence | NO |
| CDR-H1 | GFSLSSYGVS | 17 |
| CDR-H2 | VIWGDGSTNYHPNLMS | 18 |
| CDR-H3 | LDY | 19 |
| CDR-L1 | RSSQSLVHRNGNTYLH | 20 |
| CDR-L2 | KVSNRFS | 21 |
| CDR-L3 | FQTTYVPNT | 22 |

In a particular embodiment, the antibody-peptide fusion protein comprises an antibody, wherein the antibody comprises a VH that comprises (a) a CDR-H1 comprising the amino acid sequence of SEQ ID NO:17, (b) a CDR-H2 comprising the amino acid sequence of SEQ ID NO:18, and (c) a CDR-H3 comprising the amino acid sequence of SEQ ID NO:19, (d) a CDR-L1 comprising the amino acid sequence of SEQ ID NO:20; (e) a CDR-L2 comprising the amino acid sequence of SEQ ID NO:21; and (f) a CDR-L3 comprising the amino acid sequence of SEQ ID NO:22, wherein the antibody is linked to a amyloid-reactive peptide via a spacer comprising an amino acid sequence selected from the group consisting of SEQ ID NOs: 23-24, 27, 83-86.

m11-1F4 VH
SEQ ID NO: 15
QVQLKESGPGLVAPSQSLSITCTVSGFSLSSYGVSWVRQPPGKGL
EWLGVIWGDGSTNYHPNLMSRLSISKDISKSQVLFKLNSLQTDDT
ATYYCVTLDYWGQGTSVTVSS
m11-1F4 VL
SEQ ID NO: 16
DVVMTQTPLSLPVSLGDQASISCRSSQSLVHRNGNTYLHWYLQKP
GQSPKLLIYKVSNRFSGVPDRFSGSGSGTDFTLKISRVEAEDLGL
YFCFQTTYVPNTFGGGTKLEIK

Also provided herein are antibody-peptide fusion proteins comprising a humanized antibody that binds to human amyloid fibrils fused to an amyloid-reactive peptide. In some embodiments, the antibody-peptide fusion protein comprises a humanized antibody as described herein. In some embodiments, the humanized antibody comprises a humanized VH and/or VL sequence derived from m11-1F4. In some embodiments, the antibody-peptide fusion protein comprises a humanized antibody as described in International Application No. PCT/US2020/060596, which is hereby incorporated by reference in its entirety. Exemplary amino acid sequences of humanized VH and VL regions are provided below in Tables 3-4. In Tables 3-4, CDR sequences are underlined, and back mutated residues and further mutations that were introduced into the humanized variants VL4 and VH9 are bolded, and italicized. Further mutations that were introduced into VL4 and VH9 are listed in the IgG column of Tables 3-4; these mutations are numbered relative to the N-terminus of the VL or VH. CDR amino acid sequences for variants of VL4 and VH9 with modified CDRs are presented in Table 5 and Table 6, as compared to VL4 and VH9, below.

TABLE 3
Amino acid sequences of humanized light chain variable region sequences

| | | SEQ |
|---|---|---|
| IgG | VL Amino Acid Sequence | ID NO |
| VL1 | DVVMTQSPLSLPVTLGQPASISCRSSQSLVHRNGNTYLHWFQQRPGQSPRRL | 32 |
| | IYKVSNRFSGVPDRESGSGSGTDFTLKISRVEAEDVGVYYCFQTTYVPNTFG | |
| | GGTKLEIK | |
| VL2 | DVVMTQSPLSLPVTLGQPASISCRSSQSLVHRNGNTYLHWYLQRPGQSPRRL | 33 |
| | IYKVSNRFSGVPDRESGSGSGTDFTLKISRVEAEDVGVYYCFQTTYVPNTFG | |
| | GGTKLEIK | |
| VL3 | DVVMTQSPLSLPVTLGQPASISCRSSQSLVHRNGNTYLHWYLQRPGQSPRLL | 34 |
| | IYKVSNRFSGVPDRFSGSGSGTDFTLKISRVEAEDVGLYFCFQTTYVPNTFG | |
| | GGTKLEIK | |
| VL4 | DVVMTQSPLSLPVTLGQPASISCRSSQSLVHRNGNTYLHWFQQRPGQSPRLL | 35 |
| | IYKVSNRFSGVPDRESGSGSGTDFTLKISRVEAEDVGVYFCFQTTYVPNTFG | |
| | GGTKLEIK | |
| VL4-N33S | DVVMTQSPLSLPVTLGQPASISCRSSQSLVHRSGNTYLHWFQQRPGQSPRLL | 36 |
| | IYKVSNRFSGVPDRFSGSGSGTDFTLKISRVEAEDVGVYFCFQTTYVPNTFG | |
| | GGTKLEIK | |
| VL4-N33Q | DVVMTQSPLSLPVTLGQPASISCRSSQSLVHRQGNTYLHWFQQRPGQSPRLL | 37 |
| | IYKVSNRFSGVPDRESGSGSGTDFTLKISRVEAEDVGVYFCFQTTYVPNTFG | |
| | GGTKLEIK | |
| VL4-N33E | DVVMTQSPLSLPVTLGQPASISCRSSQSLVHREGNTYLHWFQQRPGQSPRLL | 38 |
| | IYKVSNRFSGVPDRESGSGSGTDFTLKISRVEAEDVGVYFCFQTTYVPNTFG | |
| | GGTKLEIK | |
| VL4-N33A | DVVMTQSPLSLPVTLGQPASISCRSSQSLVHRAGNTYLHWFQQRPGQSPRLL | 39 |
| | IYKVSNRFSGVPDRESGSGSGTDFTLKISRVEAEDVGVYFCFQTTYVPNTFG | |
| | GGTKLEIK | |
| VL4-N33H | DVVMTQSPLSLPVTLGQPASISCRSSQSLVHRHGNTYLHWFQQRPGQSPRLL | 40 |
| | IYKVSNRFSGVPDRESGSGSGTDFTLKISRVEAEDVGVYFCFQTTYVPNTFG | |
| | GGTKLEIK | |
| VL4-G34A | DVVMTQSPLSLPVTLGQPASISCRSSQSLVHRAGNTYLHWFQQRPGQSPRLL | 41 |
| | IYKVSNRFSGVPDRFSGSGSGTDFTLKISRVEAEDVGVYFCFQTTYVPNTFG | |
| | GGTKLEIK | |
| VL4-G34V | DVVMTQSPLSLPVTLGQPASISCRSSQSLVHRVGNTYLHWFQQRPGQSPRLL | 42 |
| | IYKVSNRFSGVPDRFSGSGSGTDFTLKISRVEAEDVGVYFCFQTTYVPNTFG | |
| | GGTKLEIK | |

TABLE 4
Amino acid sequences of humanized heavy chain variable region sequences

| | | SEQ |
|---|---|---|
| IgG | VH Amino Acid Sequence | ID NO |
| VH1 | QVQLQESGPGLVKPSETLSLTCTVSGFSLSSYGVSWIRQPPGKGLEWIGVIW | 43 |
| | GDGSTNYHPNLMSRVTISVDTSKNQFSLKLSSVTAADTAVYYCARLDYWGQG | |
| | TSVTVSS | |
| VH2 | QVQLQESGPGLVKPSETLSLTCTVSGFSLSSYGVSWVRQPPGKGLEWLGVIW | 44 |
| | GDGSTNYHPNLMSRVTISVDTSKNQFSLKLSSVTAADTAVYYCARLDYWGQG | |
| | TSVTVSS | |
| VH3 | QVQLQESGPGLVKPSETLSLTCTVSGFSLSSYGVSWIRQPPGKGLEWIGVIW | 45 |
| | GDGSTNYHPNLMSRLSISVDTSKNQFSLKLSSVTAADTATYYCVTLDYWGQG | |
| | TSVTVSS | |
| VH4 | QVQLQESGPGLVKPSETLSLTCTVSGFSLSSYGVSWVRQPPGKGLEWLGVIW | 46 |
| | GDGSTNYHPNLMSRLSISVDTSKNQFSLKLSSVTAADTAVYYCARLDYWGQG | |
| | TSVTVSS | |
| VH5 | QVQLQESGPGLVKPSETLSLTCTVSGFSLSSYGVSWVRQPPGKGLEWLGVIW | 47 |
| | GDGSTNYHPNLMSRLSISVDTSKNQFSLKLSSVTAADTAVYYCVTLDYWGQG | |
| | TSVTVSS | |
| VH6 | QVQLQESGPGLVKPSETLSLTCTVSGFSLSSYGVSWVRQPPGKGLEWLGVIW | 48 |
| | GDGSTNYHPNLMSRLSISKDTSKNQFSLKLSSVTAADTATYYCVTLDYWGQG | |
| | TSVTVSS | |
| VH7 | QVQLQESGPGLVKPSETLSLTCTVSGFSLSSYGVSWIRQPPGKGLEWIGVIW | 49 |
| | GDGSTNYHPNLMSRVTISKDTSKNQVLLKLSSVTAADTAVYYCVTLDYWGQG | |
| | TSVTVSS | |
| VH8 | QVQLQESGPGLVKPSETLSLTCTVSGFSLSSYGVSWIRQPPGKGLEWIGVIW | 50 |
| | GDGSTNYHPNLMSRVTISKDTSKSQFSLKLSSVTAADTAVYYCVTLDYWGQG | |
| | TSVTVSS | |
| VH9 | QVQLQESGPGLVKPSETLSLTCTVSGFSLSSYGVSWIRQPPGKGLEWLGVIW | 51 |
| | GDGSTNYHPNLMSRVTISVDTSKSQVLFKLSSVTAADTAVYYCATLDYWGQG | |
| | TSVTVSS | |
| VH10 | QVQLQESGPGLVKPSETLSLTCTVSGFSLSSYGVSWIRQPPGKGLEWLGVIW | 52 |
| | GDGSTNYHPNLMSRLSISKDTSKSQVLLKLSSVTAADTAVYYCVTLDYWGQG | |
| | TSVTVSS | |
| VH9-D54S | QVQLQESGPGLVKPSETLSLTCTVSGFSLSSYGVSWIRQPPGKGLEWLGVIW | 53 |
| | GSGSTNYHPNLMSRVTISVDTSKSQVLFKLSSVTAADTAVYYCATLDYWGQG | |
| | TSVTVSS | |
| VH9-D54Q | QVQLQESGPGLVKPSETLSLTCTVSGFSLSSYGVSWIRQPPGKGLEWLGVIW | 54 |
| | GQGSTNYHPNLMSRVTISVDTSKSQVLFKLSSVTAADTAVYYCATLDYWGQG | |
| | TSVTVSS | |
| VH9-D54E | QVQLQESGPGLVKPSETLSLTCTVSGFSLSSYGVSWIRQPPGKGLEWLGVIW | 55 |
| | GEGSTNYHPNLMSRVTISVDTSKSQVLFKLSSVTAADTAVYYCATLDYWGQG | |
| | TSVTVSS | |
| VH9-D54A | QVQLQESGPGLVKPSETLSLTCTVSGFSLSSYGVSWIRQPPGKGLEWLGVIW | 56 |
| | GAGSTNYHPNLMSRVTISVDTSKSQVLFKLSSVTAADTAVYYCATLDYWGQG | |
| | TSVTVSS | |
| VH9-D54H | QVQLQESGPGLVKPSETLSLTCTVSGFSLSSYGVSWIRQPPGKGLEWLGVIW | 57 |
| | GHGSTNYHPNLMSRVTISVDTSKSQVLFKLSSVTAADTAVYYCATLDYWGQG | |
| | TSVTVSS | |
| VH9-G55A | QVQLQESGPGLVKPSETLSLTCTVSGFSLSSYGVSWIRQPPGKGLEWLGVIW | 58 |
| | GDASTNYHPNLMSRVTISVDTSKSQVLFKLSSVTAADTAVYYCATLDYWGQG | |
| | TSVTVSS | |
| VH9-G55V | QVQLQESGPGLVKPSETLSLTCTVSGFSLSSYGVSWIRQPPGKGLEWLGVIW | 59 |
| | GDVSTNYHPNLMSRVTISVDTSKSQVLFKLSSVTAADTAVYYCATLDYWGQG | |
| | TSVTVSS | |
| VH9-M64V | QVQLQESGPGLVKPSETLSLTCTVSGFSLSSYGVSWIRQPPGKGLEWLGVIW | 60 |
| | GDGSTNYHPNLVSRVTISVDTSKSQVLFKLSSVTAADTAVYYCATLDYWGQG | |
| | TSVTVSS | |
| VH9-M64I | QVQLQESGPGLVKPSETLSLTCTVSGFSLSSYGVSWIRQPPGKGLEWLGVIW | 61 |
| | GDGSTNYHPNLISRVTISVDTSKSQVLFKLSSVTAADTAVYYCATLDYWGQG | |
| | TSVTVSS | |
| VH9-M64L | QVQLQESGPGLVKPSETLSLTCTVSGFSLSSYGVSWIRQPPGKGLEWLGVIW | 62 |
| | GDGSTNYHPNLLSRVTISVDTSKSQVLFKLSSVTAADTAVYYCATLDYWGQG | |
| | TSVTVSS | |
| VH9-M64A | QVQLQESGPGLVKPSETLSLTCTVSGFSLSSYGVSWIRQPPGKGLEWLGVIW | 63 |
| | GDGSTNYHPNLASRVTISVDTSKSQVLFKLSSVTAADTAVYYCATLDYWGQG | |
| | TSVTVSS | |

TABLE 5
Amino acid sequences of VL4 CDRs

| | CDR-L1 | | CDR-L2 | | CDR-L3 | |
|---|---|---|---|---|---|---|
| | Amino | SEQ | Amino | SEQ | Amino | SEQ |
| | Acid | ID | Acid | ID | Acid | ID |
| IgG | Sequence | NO | Sequence | NO | Sequence | NO |
| VL4 | RSSQSLVH | 20 | KVSNRFS | 21 | FQTTYVPNT | 22 |
| | RNGNTYLH | | | | | |
| VL4- | RSSQSLVH | 64 | KVSNRFS | 21 | FQTTYVPNT | 22 |
| N33S | RSGNTYLH | | | | | |
| VL4- | RSSQSLVH | 65 | KVSNRES | 21 | FQTTYVPNT | 22 |
| N33Q | RQGNTYLH | | | | | |
| VL4- | RSSQSLVH | 66 | KVSNRFS | 21 | FQTTYVPNT | 22 |
| N33E | REGNTYLH | | | | | |
| VL4- | RSSQSLVH | 67 | KVSNRES | 21 | FQTTYVPNT | 22 |
| N33A | RAGNTYLH | | | | | |
| VL4- | RSSQSLVH | 68 | KVSNRES | 21 | FQTTYVPNT | 22 |
| N33H | RHGNTYLH | | | | | |
| VL4- | RSSQSLVH | 69 | KVSNRFS | 21 | FQTTYVPNT | 22 |
| G34A | RNANTYLH | | | | | |
| VL4- | RSSQSLVH | 70 | KVSNRFS | 21 | FQTTYVPNT | 22 |
| G34V | RNVNTYLH | | | | | |

TABLE 6
Amino acid sequences of VH9 CDRs

| | CDR-H1 | | CDR-H2 | | CDR-H3 | |
|---|---|---|---|---|---|---|
| | Amino | SEQ | Amino | SEQ | Amino | SEQ |
| | Acid | ID | Acid | ID | Acid | ID |
| IgG | Sequence | NO | Sequence | NO | Sequence | NO |
| VH9 | GFSLSSYGVS | 17 | VIWGDGSTNYHPNLMS | 18 | LDY | 19 |
| VH9- | GFSLSSYGVS | 17 | VIWGSGSTNYHPNLMS | 71 | LDY | 19 |
| D54S | | | | | | |
| VH9- | GFSLSSYGVS | 17 | VIWGQGSTNYHPNLMS | 72 | LDY | 19 |
| D54Q | | | | | | |
| VH9- | GFSLSSYGVS | 17 | VIWGEGSTNYHPNLMS | 73 | LDY | 19 |
| D54E | | | | | | |
| VH9- | GFSLSSYGVS | 17 | VIWGAGSTNYHPNLMS | 74 | LDY | 19 |
| D54A | | | | | | |
| VH9- | GFSLSSYGVS | 17 | VIWGHGSTNYHPNLMS | 75 | LDY | 19 |
| D54H | | | | | | |
| VH9- | GFSLSSYGVS | 17 | VIWGDASTNYHPNLMS | 76 | LDY | 19 |
| G55A | | | | | | |
| VH9- | GFSLSSYGVS | 17 | VIWGDVSTNYHPNLMS | 77 | LDY | 19 |
| G55V | | | | | | |
| VH9- | GFSLSSYGVS | 17 | VIWGDGSTNYHPNLVS | 78 | LDY | 19 |
| M64V | | | | | | |
| VH9- | GFSLSSYGVS | 17 | VIWGDGSTNYHPNLIS | 79 | LDY | 19 |
| M64I | | | | | | |
| VH9- | GFSLSSYGVS | 17 | VIWGDGSTNYHPNLLS | 80 | LDY | 19 |
| M64L | | | | | | |
| VH9- | GFSLSSYGVS | 17 | VIWGDGSTNYHPNLAS | 81 | LDY | 19 |
| M64A | | | | | | |

In some embodiments, the antibody-peptide fusion protein comprises a humanized antibody comprising a light chain variable region (VL) and a heavy chain variable region (VH), wherein the VL comprises a CDR-L1 comprising the amino acid sequence set forth in SEQ ID NO:20, a CDR-L2 comprising the amino acid sequence set forth in SEQ ID NO:21, and a CDR-L3 comprising the amino acid sequence set forth in SEQ ID NO:22, and the VH comprises a CDR-H1 comprising the amino acid sequence set forth in SEQ ID NO:17, a CDR-H2 comprising the amino acid sequence set forth in SEQ ID NO:18, and a CDR-H3 comprising the amino acid sequence set forth in SEQ ID NO:19. In some embodiments, the humanized antibody comprises one, two, three, four, five, or six CDRs of an antibody as shown in Table 2. In some embodiments, the humanized antibody comprises a CDR-H1, a CDR-H2, and a CDR-H3, respectively comprising the amino acid sequences of a CDR-H1, a CDR-H2, and a CDR-H3 of a VH having the sequence set forth in SEQ ID NO:15; and a CDR-L1, a CDR-L2, and a CDR-L3, respectively comprising the amino acid sequences of a CDR-L1, a CDR-L2, and a CDR-L3 of a VL having the sequence set forth in SEQ ID NO:16. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86.

In some embodiments, antibody-peptide fusion protein comprises a humanized antibody comprising a VL comprising a CDR-L1 comprising the amino acid sequence set forth in SEQ ID NOs:64-70, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence set forth in SEQ ID NO:18, and a CDR-H3 comprising the amino acid sequence set forth in SEQ ID NO:19. In some embodiments, the antibody-peptide fusion protein comprises a humanized antibody, wherein the humanized antibody comprises a VL comprising a CDR-L1 comprising the amino acid sequence set forth in SEQ ID NO: 20; a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence set forth in SEQ ID NOs: 71-81; and a CDR-H3 comprising the amino acid sequence set forth in SEQ ID NO:19. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86.

In some embodiments, antibody-peptide fusion protein comprises a humanized antibody comprising a VL comprising a CDR-L1 comprising the amino acid sequence selected from the group consisting of SEQ ID NOs: 64-70, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence set forth in SEQ ID NO:71, and a CDR-H3 comprising the amino acid sequence set forth in SEQ ID NO:19. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86.

In some embodiments, antibody-peptide fusion protein comprises a humanized antibody comprising a VL comprising a CDR-L1 comprising the amino acid sequence selected from the group consisting of SEQ ID NOs: 64-70, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence set forth in SEQ ID NO:72, and a CDR-H3 comprising the amino acid sequence set forth in SEQ ID NO:19. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86.

In some embodiments, antibody-peptide fusion protein comprises a humanized antibody comprising a VL comprising a CDR-L1 comprising the amino acid sequence selected from the group consisting of in SEQ ID NOs: 64-70, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86.

In some embodiments, antibody-peptide fusion protein comprises a humanized antibody comprising a VL comprising a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence selected from the group consisting of SEQ ID NOs: 71-81, and a CDR-H3 comprising the amino acid sequence set forth in SEQ ID NO:19.

In some embodiments, antibody-peptide fusion protein comprises a humanized antibody comprising a VL comprising a CDR-L1 comprising the amino acid sequence set forth in SEQ ID NO:65, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence selected from the group consisting of SEQ ID NOs: 71-81, and a CDR-H3 comprising the amino acid sequence set forth in SEQ ID NO:19.

In some embodiments, antibody-peptide fusion protein comprises a humanized antibody comprising a VL comprising a CDR-L1 comprising the amino acid sequence set forth in SEQ ID NO:66, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence selected from the group consisting of SEQ ID NOs: 71-81, and a CDR-H3 comprising the amino acid sequence set forth in SEQ ID NO:19.

In some embodiments, antibody-peptide fusion protein comprises a humanized antibody comprising a VL comprising a CDR-L1 comprising the amino acid sequence set forth in SEQ ID NO:67, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence selected from the group consisting of SEQ ID NOs: 71-81, and a CDR-H3 comprising the amino acid sequence set forth in SEQ ID NO:19.

In some embodiments, antibody-peptide fusion protein comprises a humanized antibody comprising a VL comprising a CDR-L1 comprising the amino acid sequence set forth in SEQ ID NO:68, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence selected from the group consisting of SEQ ID NOs: 71-81, and a CDR-H3 comprising the amino acid sequence set forth in SEQ ID NO:19.

In some embodiments, antibody-peptide fusion protein comprises a humanized antibody comprising a VL comprising a CDR-L1 comprising the amino acid sequence set forth in SEQ ID NO:69, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence selected from the group consisting of SEQ ID NOs: 71-81, and a CDR-H3 comprising the amino acid sequence set forth in SEQ ID NO:19. acid sequence selected from the group consisting of SEQ ID NOs: 83-86.

In some embodiments, antibody-peptide fusion protein comprises a humanized antibody comprising a VL comprising a CDR-L1 comprising the amino acid sequence set forth in SEQ ID NO:70, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence selected from the group consisting of SEQ ID NOs: 71-81, and a CDR-H3 comprising the amino acid sequence set forth in SEQ ID NO:19.

In some embodiments, antibody-peptide fusion protein comprises a humanized antibody comprising a VL comprising a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22, and a VH comprising a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19. In some embodiments, the antibody-peptide fusion protein is a full length antibody. In some embodiments, the antibody-peptide fusion protein has an IgG1 isotype. In some embodiments, the antibody-peptide fusion protein comprises a light chain comprising from N- to C-terminus in order a VL, a CL, a spacer, and an amyloid reactive peptide. In some embodiments, the amyloid-reactive peptide is fused to the C-terminus of the light chain via a spacer.

In some embodiments, the antibody-peptide fusion protein comprises a humanized antibody comprising the amino acid sequence of a VL as shown in Table 3. In some embodiments, the antibody-peptide fusion protein comprises a humanized antibody, wherein the humanized antibody comprises a VL selected from the group consisting of VL2, VL3, VL4, VL4-N33S, VL4-N33Q, VL4-N33E, VL4-N33A, VL4-N33H, VL4-G34A, or VL4-G34V, as shown in Table 3. In some embodiments, the VL comprises an amino acid sequence set forth in the group consisting of SEQ ID NOs: 32-42. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86. In some embodiments, the amyloid-reactive peptide comprises the amino acid sequence selected from the group consisting of SEQ ID NOs: 1-13.

In some embodiments, the antibody-peptide fusion protein comprises a humanized antibody comprising the amino acid sequence of a VH as shown in Table 4. In some embodiments, the antibody-peptide fusion protein comprises a humanized antibody, wherein the humanized antibody comprises a VH selected from the group consisting of VH2, VH3, VH4, VH5, VH6, VH7, VH8, VH9, VH10, VH9-D54S, VH9-D54Q, VH9-D54E, VH9-D54A, VH9-D54H, VH9-G55A, VH9-G55V, VH9-M64V, VH9-M64I, VH9-M64L, or VH9-M64A, as shown in Table 4. In some embodiments, the VH comprises an amino acid sequence set forth in the group consisting of SEQ ID NOs: 43-63. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86. In some embodiments, the amyloid-reactive peptide comprises the amino acid sequence selected from the group consisting of SEQ ID NOs: 1-13.

In some embodiments, the antibody-peptide fusion protein comprises a humanized antibody comprising a VL comprising an amino acid sequence set forth in SEQ ID NO:34, and a VH comprising an amino acid sequence set forth in SEQ ID NO:48.

In some embodiments, the antibody-peptide fusion protein comprises a humanized antibody comprising a VL comprising an amino acid sequence set forth in SEQ ID NO:35, and a VH comprising an amino acid sequence set forth in SEQ ID NO:51.

In some embodiments, the antibody-peptide fusion protein comprises a humanized antibody comprising a VL comprising an amino acid sequence set forth in SEQ ID NO:35, and a VH comprising an amino acid sequence set forth in SEQ ID NO:52

In some embodiments, the antibody-peptide fusion protein comprises a humanized antibody comprising a VL comprising an amino acid sequence set forth in SEQ ID NO:35, and a VH comprising an amino acid sequence set forth in SEQ ID NO:50.

In some embodiments, the antibody-peptide fusion protein comprises a humanized antibody comprising a VL comprising an amino acid sequence set forth in SEQ ID NO:35, and a VH comprising an amino acid sequence set forth in SEQ ID NO:49.

In some embodiments, the antibody-peptide fusion protein comprises a humanized antibody comprising a VL comprising an amino acid sequence set forth in SEQ ID NO:36, and a VH comprising an amino acid sequence set forth in SEQ ID NO:55. In some embodiments, the antibody-peptide fusion protein comprises an antibody linked to an amyloid-reactive peptide via a spacer. In some embodiments, the amyloid-reactive peptide is fused to the C-terminus of the light chain via a spacer. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86. In some embodiments, the amyloid-reactive peptide is linked to the antibody via a spacer comprising an amino acid sequence selected from the group consisting of SEQ ID NO:83 and SEQ ID NO:86. In some embodiments, the amyloid-reactive peptide is linked to the antibody via a spacer comprising an amino acid sequence set forth in SEQ ID NO:83. In some embodiments, the amyloid-reactive peptide comprises the amino acid sequence selected from the group consisting of SEQ ID NOs: 1-13. In some embodiments, the amyloid-reactive peptide comprises the amino acid sequence set forth in SEQ ID NO:1 or SEQ ID NO:2. In some embodiments, the amyloid-reactive peptide comprises the amino acid sequence set forth in SEQ ID NO:2. In some embodiments, the antibody-peptide fusion protein comprises an antibody linked to an amyloid-reactive peptide set forth in SEQ ID NO:2 via a spacer comprising an amino acid sequence set forth in SEQ ID NO:83.

In some embodiments, the antibody-peptide fusion protein comprises a humanized antibody comprising the VL of VL4 as shown in Table 3, and the VH of VH9 as shown in Table 4. In some embodiments, the antibody-peptide fusion protein comprises a humanized antibody comprising a VL comprising an amino acid sequence set forth in SEQ ID NO:35, and a VH comprising an amino acid sequence set forth in SEQ ID NO:51. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86. In some embodiments, the amyloid-reactive peptide comprises the amino acid sequence selected from the group consisting of SEQ ID NOs: 1-13.

In some embodiments, the antibody-peptide fusion protein comprises a humanized antibody comprising the VL of VL4-N33S as shown in Table 3, and the VH of VH9-D54E as shown in Table 4. In some embodiments, the antibody-peptide fusion protein comprises a humanized antibody comprising a VL comprising an amino acid sequence set forth in SEQ ID NO:36, and a VH comprising an amino acid sequence set forth in SEQ ID NO:55. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86. In some embodiments, the amyloid-reactive peptide comprises the amino acid sequence selected from the group consisting of SEQ ID NOs: 1-13.

In some embodiments, the antibody-peptide fusion protein comprises a humanized antibody, wherein the humanized antibody comprises a light chain. In some embodiments, the amyloid-reactive peptide is fused to the C-terminus of the light chain. In some embodiments, the antibody-peptide fusion protein comprises a humanized antibody, wherein the humanized antibody comprises a light chain, wherein the amyloid-reactive peptide is fused to the C-terminus of the light chain by a spacer. In some embodiments, the spacer is a peptide spacer. In some embodiments, the spacer is a flexible spacer. In some embodiments, the space comprises glycine and serine residues. In some embodiments, the spacer comprises the amino acid sequence GGGYS. In some embodiments, the spacer comprises the amino acid sequence set forth in SEQ ID NO:27. In some embodiments, the spacer is a rigid spacer. In some embodiments, the spacer is uncharged. In some embodiments, the spacer comprises an amino acid sequence set forth in SEQ ID NOs: 83-86.

In certain embodiments, the antibody-peptide fusion protein may include spacer sequences of amino acids between the C-terminus of the light chain and the amyloid-reactive peptide. In certain embodiments, the peptide-Ig conjugates may include spacer sequences of amino acids between the N-terminal of the peptide and a leader sequence required for secretion of the Ig-peptide from cells expressing the reagent. In some embodiments, the spacer is a flexible spacer. In some embodiments, the spacer is a rigid spacer peptide. In some embodiments, the spacer is uncharged. In some embodiments a spacer peptide may comprise or consist of from about 3 to about 55 amino acids. The spacer peptides of the present invention may comprise or consist of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. As used herein, a nucleic acid sequence or amino acid sequence is “adjacent” to another nucleic acid sequence or amino acid sequence if such nucleic acid sequences or amino acid sequences are close to each other in sequence. For example, two nucleic acid sequences can be adjacent to each other as described herein but still include an intervening spacer sequence. In some embodiments, the spacer peptide comprises an amino acid sequence as set forth in Table 7, below. In some embodiments, the spacer comprises an amino acid sequence set forth in SEQ ID NOs: 23-24, 27, and 83-86.

TABLE 7
Exemplary Spacer Sequences

| | Amino | |
|---|---|---|
| | Acid | |
| Description | Sequence | SEQ ID NO |
| Spacer 1 | AQAGQAGQ | SEQ ID NO: 23 |
| | AQGGGYS | |
| Spacer 2 | VTPTV | SEQ ID NO: 24 |
| Spacer 3 | GGGYS | SEQ ID NO: 27 |
| Short, rigid | VSPSV | SEQ ID NO: 83 |
| spacer | | |
| Long, rigid | VSPSVVS | SEQ ID NO: 84 |
| spacer | PSV | |
| Flexible, | GGSGG | SEQ ID NO: 85 |
| short spacer | | |
| Flexible, | GGGGSGG | SEQ ID NO: 86 |
| long spacer | GGS | |

In some embodiments, one or more of the peptides shown in Table 1 can be linked to a humanized antibody or functional fragment thereof through the C- or N-terminus of the light chain protein or the C- or N-terminus of the heavy chain, thereby forming an antibody-peptide fusion protein comprising a humanized antibody. That is, any of the sequences identified in Table 1 can be linked to the heavy or light chain of the humanized antibody or functional fragment thereof independently or simultaneously to form an antibody-peptide fusion protein. For example, two of the amyloid-reactive peptides can be linked with a single antibody, such by linking the amyloid-reactive peptide amino acid sequences to the N-terminus of a humanized antibody light chain, or linking the amyloid-reactive peptide amino acid sequences to the C-terminus of a humanized antibody light chain, or linking the amyloid-reactive peptide amino acid sequences to the C-terminus of a humanized antibody heavy chain.

In some embodiments, the antibody-peptide fusion protein comprises a light chain further comprising a light chain constant region (e.g., comprising a CL1), and a heavy chain comprising a heavy chain constant region (e.g., comprising a CH1, a CH2, and a CH3). In some embodiments, the antibody-peptide fusion protein comprises two light chains and two heavy chains.

In some embodiments, the antibody-peptide fusion protein comprises a light chain comprising, from N- to C-terminus, a light chain and an amyloid-reactive peptide. In some embodiments, the light chain comprises, from N- to C-terminus, a VL and a CL. In some embodiments, the VL is any one of the VLs described herein. In some embodiments, the antibody-peptide fusion protein comprises a heavy chain comprising, from N- to C-terminus, a VH, a CH1, a CH2, and a CH3. In some embodiments, the VH is any one of the VHs described herein. In some embodiments, the antibody-peptide fusion protein comprises a first and second light chain comprising, from the N-terminal to C-terminal direction a variable light chain region, a constant light chain region, a spacer, and an amyloid reactive peptide and a first and second heavy chain comprising, from N- to C-terminus, a VH, a CH1, a CH2, and a CH3, wherein the CH2 and the CH3 of the first and second heavy chain form a dimer.

In some embodiments, the antibody-peptide fusion protein comprises a light chain comprising, from N- to C-terminus, a light chain, a spacer peptide, and an amyloid-reactive peptide. In some embodiments, the spacer peptide comprises the amino acid sequence of SEQ ID NO:23. In some embodiments, the spacer peptide comprises the amino acid sequence of SEQ ID NO:27. In some embodiments, the spacer peptide comprises an amino acid sequence of SEQ ID NOs: 83-86. In some embodiments, the light chain comprises, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CH1, a CH2, and a CH3. In some embodiments, the VH is any one of the VHs described herein. In some embodiments, the antibody-peptide fusion protein comprises a first and second light chain comprising, from N- to C-terminus, a VL, a CL1, a spacer, and an amyloid-reactive peptide, and a first and second heavy chain comprising, from N- to C-terminus, a VH, a CH1, a CH2, and a CH3, wherein the CH2 and the CH3 of the first and second heavy chain form a dimer.

In some embodiments, the antibody-peptide fusion protein comprises, from N- to C-terminus, a secretory leader peptide, a first spacer peptide, an amyloid-reactive peptide, a second spacer peptide, and a light chain. In some embodiments, the first spacer peptide comprises the amino acid sequence of SEQ ID NO:23. In some embodiments, the first spacer peptide comprises the amino acid sequence of SEQ ID NO:27. In some embodiments, the second spacer peptide comprises the amino acid sequence of SEQ ID NO:24. In some embodiments, the first and/or second spacer peptide comprises an amino acid sequence of SEQ ID NOs: 83-86. In some embodiments, the light chain comprises, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CH1, a CH2, and a CH3. In some embodiments, the VH is any one of the VHs described herein. In some embodiments, the VH is any one of the VHs described herein.

In some embodiments, the antibody-peptide fusion protein comprises, from N- to C-terminus, a secretory leader peptide, a first spacer peptide, a light chain, a second spacer peptide, and an amyloid-reactive peptide. In some embodiments, the first spacer peptide comprises the amino acid sequence of SEQ ID NO:23. In some embodiments, the first spacer peptide comprises the amino acid sequence of SEQ ID NO:27. In some embodiments, the second spacer peptide comprises the amino acid sequence of SEQ ID NO:24. In some embodiments, the first and/or second spacer peptide comprises an amino acid sequence of SEQ ID NOs: 83-86. In some embodiments, the light chain comprises, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CH1, a CH2, and a CH3. In some embodiments, the VH is any one of the VHs described herein. In some embodiments, the VH is any one of the VHs described herein.

In some embodiments, the antibody-peptide fusion protein comprises a light chain comprising, from N- to C-terminus, a VL and a CL1. In some embodiments, the VL is any one of the VLs described herein. In some embodiments, the antibody-peptide fusion protein comprises a heavy chain comprising, from N- to C-terminus, an amyloid-reactive peptide, VH, a CH1, a CH2, and a CH3. In some embodiments, the VH is any one of the VHs described herein. In some embodiments, the antibody-peptide fusion protein comprises a first and second light chain comprising, from N- to C-terminus, a VL, and a CL1, and a first and second heavy chain comprising, from N- to C-terminus, an amyloid-reactive peptide, a VH, a CH1, a CH2, and a CH3, wherein the CH2 and the CH3 of the first and second heavy chain form a dimer.

In some embodiments, the antibody-peptide fusion protein comprises a light chain comprising, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CH1, a CH2, a CH3, and an amyloid-reactive peptide. In some embodiments, the VH is any one of the VHs described herein. In some embodiments, the antibody-peptide fusion protein comprises a first and second light chain comprising, from N- to C-terminus, a VL, and a CL1, and a first and second heavy chain comprising, from N- to C-terminus, a VH, a CH1, a CH2, a CH3, and an amyloid-reactive peptide, wherein the CH2 and the CH3 of the first and second heavy chain form a dimer.

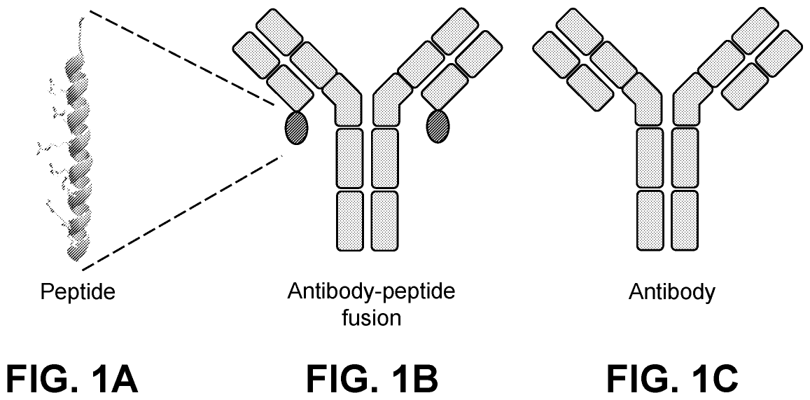

In some embodiments the antibody-peptide fusion comprises a light chain comprising in N-terminal to C-terminal direction a variable light chain region, a constant light chain region, a spacer, and an amyloid reactive peptide. In some embodiments, the amyloid-reactive peptide comprises the amino acid sequence of SEQ ID NO:2. In some embodiments, the spacer comprises the amino acid sequences of SEQ ID NO:83. In some embodiments, the light chain comprises a VL comprising the amino acid sequence of SEQ ID NO:36. In some embodiments, the heavy chain comprises a VH comprising the amino acid sequence of SEQ ID NO:55. In some embodiments, the antibody-peptide fusion protein comprises a light chain comprises an amino acid set forth in SEQ ID NO:89, and a heavy chain comprises an amino acid sequence set forth in SEQ ID NO:91. In some embodiments, the antibody-peptide fusion protein comprises a first polypeptide and a second polypeptide comprising an amyloid-reactive peptide linked to the C-terminus of a light chain of an antibody that binds to a human amyloid fibrils, and a third and a fourth polypeptide comprising a heavy chain of an antibody that binds to a human amyloid fibrils, wherein the first polypeptide and second polypeptide comprise the amino acid set forth in SEQ ID NO:89, and the third and fourth polypeptide comprise the amino acid sequence set forth in SEQ ID NO:91. In some embodiments, the antibody-peptide fusion protein comprises a structure as shown in FIG. 1C.

In some embodiments, the antibody-peptide fusion protein comprises a light chain comprising, from N- to C-terminus, a light chain, a spacer peptide, and an amyloid-reactive peptide. In some embodiments, the amyloid-reactive peptide comprises the amino acid sequence of SEQ ID NO:2. In some embodiments, the spacer comprises the amino acid sequences of SEQ ID NO:86. In some embodiments, the light chain comprises a VL comprising the amino acid sequence of SEQ ID NO:36. In some embodiments, the heavy chain comprises a VH comprising the amino acid sequence of SEQ ID NO:55. In some embodiments, the antibody-peptide fusion protein comprises a light chain comprises an amino acid set forth in SEQ ID NO:90, and a heavy chain comprises an amino acid sequence set forth in SEQ ID NO:91. In some embodiments, the antibody-peptide fusion protein comprises a first polypeptide and a second polypeptide comprising an amyloid-reactive peptide linked to the C-terminus of a light chain of an antibody that binds to human amyloid fibrils, and a third and a fourth polypeptide comprising a heavy chain of an antibody that binds to human amyloid fibrils, wherein the first polypeptide and second polypeptide comprise the amino acid set forth in SEQ ID NO:90, and the third and fourth polypeptide comprise the amino acid sequence set forth in SEQ ID NO:91. In some embodiments, the antibody-peptide fusion protein comprises a structure as shown in FIG. 1B.

In some embodiments, the antibody-peptide fusion protein comprises an antibody that binds to amyloid fibrils comprising a first polypeptide and a second polypeptide each comprising a light chain of the antibody, and a third and a forth polypeptide each comprising a heavy chain of the antibody. In some embodiments, the antibody-peptide fusion protein comprises an amyloid-reactive peptide that is linked to the N-terminus or the C-terminus of the light chain or the heavy chain. In some embodiments, the first polypeptide and second polypeptide comprise the amino acid set forth in SEQ ID NO:87, and the third and fourth polypeptide comprise the amino acid sequence set forth in SEQ ID NO:91. In some embodiments, the first polypeptide and second polypeptide comprise the amino acid set forth in SEQ ID NO:88, and the third and fourth polypeptide comprise the amino acid sequence set forth in SEQ ID NO:92. In some embodiments, the first polypeptide and second polypeptide comprise the amino acid set forth in SEQ ID NO:89, and the third and fourth polypeptide comprise the amino acid sequence set forth in SEQ ID NO:91. In some embodiments, the first polypeptide and second polypeptide comprise the amino acid set forth in SEQ ID NO:90, and the third and fourth polypeptide comprise the amino acid sequence set forth in SEQ ID NO:91. In some embodiments, the antibody-peptide fusion protein comprises an antibody linked to an amyloid-reactive peptide via a spacer comprising an amino acid sequence selected from the group consisting of SEQ ID NOs: 23-24, 27, 83-86. In some embodiments, the amyloid-reactive peptide is linked to the antibody via a spacer comprising an amino acid sequence selected from the group consisting of SEQ ID NOs: 83-86. In some embodiments, the amyloid-reactive peptide comprises the amino acid sequence selected from the group consisting of SEQ ID NOs: 1-13. In some embodiments, the amyloid-reactive peptide comprises the amino acid sequence set forth in SEQ ID NO:1 or SEQ ID NO:2. In some embodiments, the amyloid-reactive peptide comprises the amino acid sequence set forth in SEQ ID NO:2. In some embodiments, the antibody-peptide fusion protein comprises an antibody linked to an amyloid-reactive peptide set forth in SEQ ID NO:2 via a spacer comprising an amino acid sequence set forth in SEQ ID NO:83.

In some embodiments, the antibody-peptide fusion protein comprises an amyloid-reactive peptide comprising the amino acid sequence set forth in SEQ ID NO:1 or SEQ ID NO:2. In some embodiments, the antibody-peptide fusion protein comprises an antibody that binds to human amyloid fibrils. In some embodiments, the antibody comprises a variable heavy chain (VH) and a variable light chain (VL) wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22. In some embodiments, the amyloid-reactive peptide and antibody are linked at the C-terminal end of the light chain. In some embodiments, the amyloid-reactive peptide is linked to the antibody via a spacer. In some embodiments the antibody-peptide fusion comprises a light chain comprising in N-terminal to C-terminal direction a variable light chain region, a constant light chain region, a spacer, and an amyloid reactive peptide. In some embodiments, the spacer comprises an amino acid sequence selected from the group consisting of SEQ ID NOs: 23-24, 27, 83-86. In some embodiments, the spacer comprises an amino acid sequence selected from the group consisting of SEQ ID NOs: 83-86. In some embodiments, the spacer comprises an amino acid sequence set forth in SEQ ID NO:83.

Exemplary amyloid-reactive peptide-antibody fusion protein amino acid sequences are provided in Table 8 and Table 9. In Table 8, the amino acid sequence of the amyloid-reactive peptide p5R (SEQ ID NO:2) is shown in bold, and spacer sequences are underlined and italicized.

TABLE 8
Antibody-peptide fusion protein light and heavy chain sequences

| | | SEQ |
|---|---|---|
| Description | Amino Acid Sequence | ID NO |
| p5R-VL4-N33S. | APGGGRAQRAQARQARQAQRAQRAQARQARQVSPSVD | 87 |
| p5R fused to N-terminus of | VVMTQSPLSLPVTLGQPASISCRSSQSLVHRSGNTYL | |
| light chain via short rigid | HWFQQRPGQSPRLLIYKVSNRFSGVPDRESGSGSGTD | |
| spacer (VSPSV). | FTLKISRVEAEDVGVYFCFQTTYVPNTFGGGTKLEIK | |
| | RTVAAPSVFIFPPSDEQLKSGTASVVCLLNNFYPREA | |
| | KVQWKVDNALQSGNSQESVTEQDSKDSTYSLSSTLTL | |
| | SKADYEKHKVYACEVTHQGLSSPVTKSENRGEC | |
| VL4-N33S | DVVMTQSPLSLPVTLGQPASISCRSSQSLVHRSGNTY | 88 |
| | LHWFQQRPGQSPRLLIYKVSNRFSGVPDRESGSGSGT | |
| | DFTLKISRVEAEDVGVYFCFQTTYVPNTFGGGTKLEI | |
| | KRTVAAPSVFIFPPSDEQLKSGTASVVCLLNNFYPRE | |
| | AKVQWKVDNALQSGNSQESVTEQDSKDSTYSLSSTLT | |
| | LSKADYEKHKVYACEVTHQGLSSPVTKSENRGEC | |
| VL4-N33S-p5R | DVVMTQSPLSLPVTLGQPASISCRSSQSLVHRSGNTY | 89 |
| p5R fused to C-terminus of | LHWFQQRPGQSPRLLIYKVSNRFSGVPDRESGSGSGT | |
| light chain via short rigid | DFTLKISRVEAEDVGVYFCFQTTYVPNTFGGGTKLEI | |
| spacer (VSPSV). | KRTVAAPSVFIFPPSDEQLKSGTASVVCLLNNFYPRE | |
| | AKVQWKVDNALQSGNSQESVTEQDSKDSTYSLSSTLT | |
| | LSKADYEKHKVYACEVTHQGLSSPVTKSFNRGECVSP | |
| | SVRAQRAQARQARQAQRAQRAQARQARQ | |
| VL4-N33S-p5R | DVVMTQSPLSLPVTLGQPASISCRSSQSLVHRSGNTY | 90 |
| p5R fused to C-terminus of | LHWFQQRPGQSPRLLIYKVSNRFSGVPDRESGSGSGT | |
| light chain via long spacer | DFTLKISRVEAEDVGVYFCFQTTYVPNTFGGGTKLEI | |
| (GGGGSGGGGS). | KRTVAAPSVFIFPPSDEQLKSGTASVVCLLNNFYPRE | |
| | AKVQWKVDNALQSGNSQESVTEQDSKDSTYSLSSTLT | |
| | LSKADYEKHKVYACEVTHQGLSSPVTKSENRGECGGG | |
| | GSGGGGSRAQRAQARQARQAQRAQRAQARQARQ | |
| VH9-D54E | QVQLQESGPGLVKPSETLSLTCTVSGFSLSSYGVSWI | 91 |
| | RQPPGKGLEWLGVIWGEGSTNYHPNLMSRVTISVDTS | |
| | KSQVLFKLSSVTAADTAVYYCATLDYWGQGTSVTVSS | |
| | ASTKGPSVFPLAPSSKSTSGGTAALGCLVKDYFPEPV | |
| | TVSWNSGALTSGVHTFPAVLQSSGLYSLSSVVTVPSS | |
| | SLGTQTYICNVNHKPSNTKVDKKVEPKSCDKTHTCPP | |
| | CPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKA | |
| | KGQPREPQVYTLPPSRDELTKNQVSLTCLVKGFYPSD | |
| | IAVEWESNGQPENNYKTTPPVLDSDGSFFLYSKLTVD | |
| | KSRWQQGNVFSCSVMHEALHNHYTQKSLSLSPGK | |
| VH9-D54E-p5R | QVQLQESGPGLVKPSETLSLTCTVSGFSLSSYGVSWI | 92 |
| p5R fused to C-terminus of | RQPPGKGLEWLGVIWGEGSTNYHPNLMSRVTISVDTS | |
| heavy chain via short rigid | KSQVLFKLSSVTAADTAVYYCATLDYWGQGTSVTVSS | |
| spacer (VSPSV) | ASTKGPSVFPLAPSSKSTSGGTAALGCLVKDYFPEPV | |
| | TVSWNSGALTSGVHTFPAVLQSSGLYSLSSVVTVPSS | |
| | SLGTQTYICNVNHKPSNTKVDKKVEPKSCDKTHTCPP | |
| | CPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKA | |
| | KGQPREPQVYTLPPSRDELTKNQVSLTCLVKGFYPSD | |
| | IAVEWESNGQPENNYKTTPPVLDSDGSFFLYSKLTVD | |
| | KSRWQQGNVFSCSVMHEALHNHYTQKSLSLSPGKVSP | |
| | SVRAQRAQARQARQAQRAQRAQARQARQ | |
| Alternative VH9-D54E | QVQLQESGPGLVKPSETLSLTCTVSGFSLSSYGVSWI | 93 |
| C-terminal lysine deletion | RQPPGKGLEWLGVIWGEGSTNYHPNLMSRVTISVDTS | |
| | KSQVLFKLSSVTAADTAVYYCATLDYWGQGTSVTVSS | |
| | ASTKGPSVFPLAPSSKSTSGGTAALGCLVKDYFPEPV | |
| | TVSWNSGALTSGVHTFPAVLQSSGLYSLSSVVTVPSS | |
| | SLGTQTYICNVNHKPSNTKVDKKVEPKSCDKTHTCPP | |
| | CPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKA | |
| | KGQPREPQVYTLPPSRDELTKNQVSLTCLVKGFYPSD | |
| | IAVEWESNGQPENNYKTTPPVLDSDGSFFLYSKLTVD | |
| | KSRWQQGNVFSCSVMHEALHNHYTQKSLSLSPG | |

TABLE 9
Antibody-peptide fusion proteins

| | SEQ ID |
|---|---|
| Description | NOS |
| VH9-D54E/p5R-VL4-N33S | 87 and 91 |
| p5R fused to N-terminus of | |
| light chain via short rigid | |
| spacer (VSPSV). | |
| VH9-D54E-p5R/VL4-N33S | 88 and 92 |
| p5R fused to C-terminus of | |
| heavy chain via short rigid | |
| spacer (VSPSV). | |
| VH9-D54E/VL4-N33S-p5R | 89 and 91 |
| p5R fused to C-terminus of | |
| light chain via short rigid | |
| spacer (VSPSV). | |
| VH9-D54E/VL4-N33S-p5R | 90 and 91 |
| p5R fused to C-terminus of | |
| light chain via long spacer | |
| (GGGGSGGGGS). | |

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83.

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) comprising a CDR-H1, CDR-H2, and CDR-H3 of a VH comprising the amino acid sequence set forth in SEQ ID NO:55, and the light chain of the antibody comprises a light chain variable region (VL) comprising a CDR-L1, CDR-L2, and CDR-L3 of a VL comprising the amino acid sequence set forth in SEQ ID NO:36, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83.

In some embodiments, the antibody that binds to amyloid fibrils comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO:89, and the third and fourth polypeptide comprise the amino acid sequence set forth in SEQ ID NO:91.

In some embodiments, the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO:89, and the third and fourth polypeptide comprise the amino acid sequence set forth in SEQ ID NO:91.

In some aspects of the methods provided herein, the antibody-peptide fusion proteins bind to amyloid deposits or fibrils. In some embodiments, the antibody-peptide fusion protein binds to one or more amyloidogenic peptides in amyloids. In some embodiments, amyloids bound by the antibody-peptide fusion proteins comprise an amyloidogenic λ6 variable domain protein (Vλ6WIL) or an amyloidogenic immunoglobulin light chain (AL), Aβ(1-40) amyloid-like fibril or an amyloidogenic AP precursor protein, or serum amyloid protein A (AA). In other embodiments, the amyloids bound by the antibody-peptide fusion protein comprise amyloidogenic forms of immunoglobulin heavy chain (AH), β₂-microglobulin (Aβ₂M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin (ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALκ4, ALλ1 other amyloidogenic peptides. The amyloidogenic peptides bound by the antibody-peptide fusion proteins can be a protein, a protein fragment, or a protein domain. In some embodiments, the amyloid deposits or amyloid fibrils comprise recombinant amyloidogenic proteins. In some embodiments, the amyloids are part of the pathology of a disease.

The amino acids forming all or a part of the amyloid-reactive peptides bound to the antibody or fragment thereof may be stereoisomers and modifications of naturally occurring amino acids, non-naturally occurring amino acids, post-translationally modified amino acids, enzymatically synthesized amino acids, derivatized amino acids, constructs or structures designed to mimic amino acids, and the like. The amino acids forming the peptides of the present invention may be one or more of the 20 common amino acids found in naturally occurring proteins, or one or more of the modified and unusual amino acids. The antibody-peptide fusion protein may be made by any technique known to those of skill in the art, including chemical synthesis or recombinant means using standard molecular biological techniques.

In some embodiments, the antibodies of the antibody-peptide fusion proteins provided herein bind specifically to amyloid light chain fibrils. In some embodiments, the amyloid-reactive peptide binds to various amyloid fibrils such as amyloidogenic λ6 variable domain protein (Vλ6WIL) or an amyloidogenic immunoglobulin light chain (AL), Aβ(1-40) amyloid-like fibril or an amyloidogenic AP precursor protein, or serum amyloid protein A (AA). In other embodiments, the amyloids bound by the antibody-peptide fusion protein comprise amyloidogenic forms of immunoglobulin heavy chain (AH), β₂-microglobulin (Aβ₂M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin (ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALκ4, ALλ1 other amyloidogenic peptides. In some embodiments, the amyloid-reactive peptide binds to heparan sulfate glycosaminoglycans. In some embodiments, the amyloid-reactive peptide is able to bind to multiple forms of amyloid. In some embodiments, the amyloid-reactive peptide has pan-amyloid binding.

In some embodiments, the amyloid deposits or fibrils bound by the antibody-peptide fusion protein are located in one or more organ. In some embodiments, the amyloid deposits are located in one or more tissue type. In some embodiments, the amyloid deposits or fibrils are located in one or more of the liver, spleen, heart, kidney, brain, muscle, pancreas, stomach, upper intestine, lower intestine, and blood. In some embodiments, the antibody-peptide fusion proteins bind to amyloid deposits or fibrils located in at least 1, 2, 3, 4, 5, 6, 7, 8, 9, or 10 different organs and/or tissue types. In some embodiments, the antibody-peptide fusion proteins bind to amyloid deposits or fibrils located in at least 1, 2, 3, 4, 5, 6, 7, 8, 9, or 10 different organs. In some embodiments, the antibody-peptide fusion proteins bind to amyloid deposits or fibrils located in at least 1, 2, 3, 4, 5, 6, 7, 8, 9, or 10 different tissue types. In some embodiments, the antibody-peptide fusion proteins exhibit pan amyloid reactivity. In some embodiments, the antibody-peptide fusion proteins exhibit reactivity toward amyloid deposits or fibrils located in the liver, spleen, heart, kidney, brain, muscle, pancreas, stomach, upper intestine, lower intestine, and/or blood.

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83, wherein the antibody-peptide fusion protein exhibits pan amyloid reactivity. In some embodiments, the antibody-peptide fusion protein exhibits reactivity toward amyloid deposits or fibrils located in the liver, spleen, heart, kidney, brain, muscle, pancreas, stomach, upper intestine, lower intestine, and/or blood.

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) comprising a CDR-H1, CDR-H2, and CDR-H3 of a VH comprising the amino acid sequence set forth in SEQ ID NO: 55, and the light chain of the antibody comprises a light chain variable region (VL) comprising a CDR-L1, CDR-L2, and CDR-L3 of a VL comprising the amino acid sequence set forth in SEQ ID NO:36, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83, wherein the antibody-peptide fusion protein exhibits pan amyloid reactivity. In some embodiments, the antibody-peptide fusion protein exhibits reactivity toward amyloid deposits or fibrils located in the liver, spleen, heart, kidney, brain, muscle, pancreas, stomach, upper intestine, lower intestine, and/or blood.

In some embodiments, the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO:89, and the third and fourth polypeptide comprise the amino acid sequence set forth in SEQ ID NO:91, wherein the antibody-peptide fusion protein exhibits pan amyloid reactivity. In some embodiments, the antibody-peptide fusion protein exhibits reactivity toward amyloid deposits or fibrils located in the liver, spleen, heart, kidney, brain, muscle, pancreas, stomach, upper intestine, lower intestine, and/or blood.

In some embodiments, the antibody-peptide fusion proteins described herein bind to amyloid deposits or fibrils with a high binding affinity. In some embodiments, the antibody-peptide fusion proteins described herein bind to amyloid substrates with a high binding affinity. In some embodiments, the binding affinity is less than 500 nM, 400 nM, 300 nM, 200 nM, 100 nM, 50 nM, 40, nM, 30 nM, 20 nM, 10 nM, 5 nM, 4 nM, 3 nM, 2 nM, or 1.5 nM. In some embodiments, the binding affinity is less than 500 nM. In some embodiments, the binding affinity is less than 100 nM. In some embodiments, the binding affinity is less than 10 nM. In some embodiments, the binding affinity is less than 1.5 nM. In some embodiments, the binding affinity is between 0.05 nM and 100 nM, between 0.1 nM and 50 nM, between 0.2 nM and 25 nM, between 0.3 nM and 10 nM, between 0.4 nM and 5 nM, between 0.5 nM and 2 nM, between 0.6 nM and 1 nM, or between 0.2 nM and 1.5 nM. In some embodiments, the binding affinity is the same or different for different amyloid substrates. In some embodiments, the binding affinity is the same or different for human amyloid substrates. In some embodiments, the binding affinity is the same or different for synthetic amyloid substrates.

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83, wherein the EC50 binding affinity is less than 10 nM, 5 nM, or 1.5 nM. In some embodiments, the EC50 binding affinity is less than 10 nM. In some embodiments, the EC50 binding affinity is less than 1.5 nM. In some embodiments, the EC50 binding affinity is the same or different for different amyloid substrates. In some embodiments, the EC50 binding affinity is the same or different for human amyloid substrates.

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) comprising a CDR-H1, CDR-H2, and CDR-H3 of a VH comprising the amino acid sequence set forth in SEQ ID NO:55, and the light chain of the antibody comprises a light chain variable region (VL) comprising a CDR-L1, CDR-L2, and CDR-L3 of a VL comprising the amino acid sequence set forth in SEQ ID NO:36, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83, wherein the EC50 binding affinity is less than 500 nM, 100 nM, 50 nM, 10 nM, 5 nM, or 1.5 nM. In some embodiments, the EC50 binding affinity is less than 10 nM. In some embodiments, the EC50 binding affinity is less than 1.5 nM. In some embodiments, the EC50 binding affinity is the same or different for different amyloid substrates. In some embodiments, the EC50 binding affinity is the same or different for human amyloid substrates.

In some embodiments, the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO:89, and the third and fourth polypeptide comprise the amino acid sequence set forth in SEQ ID NO:91, wherein the EC50 binding affinity is less than 500 nM, 100 nM, 50 nM, 10 nM, 5 nM, or 1.5 nM. In some embodiments, the EC50 binding affinity is less than 10 nM. In some embodiments, the EC50 binding affinity is less than 1.5 nM. In some embodiments, the EC50 binding affinity is the same or different for different amyloid substrates. In some embodiments, the EC50 binding affinity is the same or different for human amyloid substrates.

As those skilled in the art will appreciate, the fragment antigen binding (or Fab region) is the head of an antibody that naturally interacts with target antigen. Components of the Fab region, for example, allow antibodies to bind to specific ligands and, through that interaction, to further activate the immune system. For IgG, IgA, IgD, IgE, and IgM antibody isotypes, the Ig is composed of two proteins, the heavy chain and light chain that interact in pairs to form an intact Ig comprising 2 heavy chains and 2 light chains. Both the heavy and light chains are further divided into variable domains and constant domains—the light and heavy variable domains comprising the Fab functional region and the heavy chains forming the fragment crystallizable (Fc) domains that interact with cell receptors and complement. The Fc regions of Ig bears a highly conserved N-glycosylation site.

In certain example embodiments, one or more of the peptides shown in Table 1 below can be linked to an antibody or functional fragment thereof through the C- or N-terminus of the light chain protein or the C- or N-terminus of the heavy chain, thereby forming an antibody-peptide fusion protein. That is, any of the sequences identified below in Table 1 can be linked to the heavy or light chain of the antibody or functional fragment thereof independently or simultaneously to form a peptide-antibody conjugate. For example, two of the amyloid-reactive peptides can be linked with a single antibody, such by joining the amyloid-reactive peptide amino acid sequences to the C-terminal of the Ig light chain proteins.

In some embodiments, the antibody is a full-length antibody comprising an Fc region. In some embodiments, the Fc is of an IgG1, IgG2, IgG3, or IgG4 isotype. In some embodiments, the antibody-peptide fusion protein comprising a humanized antibody promotes an Fc-mediated antibody effector function. In some embodiments, the antibody-peptide fusion protein comprising a humanized antibody promotes antibody-dependent cellular phagocytosis and inhibits or limits amyloid fibril growth.

In some embodiments, the antibody-peptide fusion protein comprising a humanized antibody of the present disclosure comprises an Fc region. In some embodiments, the Fc is of an IgG1, IgG2, IgG3, or IgG4 isotype. In some embodiments, the antibody-peptide fusion protein comprising a humanized antibody promotes an Fc-mediated antibody effector function. In some embodiments, the antibody-peptide fusion protein comprising a humanized antibody reduces or prevents amyloid fibril growth.

In some embodiments, the antibody-peptide fusion protein binds to human amyloid fibrils with a dissociation constant (Kd) that is less than about 100, 10, 1, 0.1, 0.01, 0.001, or 0.0001 μM. In some embodiments, the antibody-peptide fusion protein binds to human amyloid fibrils with a Kd that is about 0.0001, 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 50, 75, or 100 μM including any value or range between these values. In some embodiments, the antibody-peptide fusion protein binds to human amyloid fibrils with a Kd that is less than 500, 100, 10, or 1 nM. In some embodiments, the antibody-peptide fusion protein binds to human amyloid fibrils with a Kd that is less than about 5, 10, 20, 30, 40, 50, 60, 70, 80, 90, 100, 250, 500, 750, 1000, 2000, or 2200 nM. In some embodiments, the antibody-peptide fusion protein binds to human amyloid fibrils with a Kd that is about 5, 10, 20, 30, 40, 50, 60, 70, 80, 90, 100, 250, 500, 750, 1000, 2000, or 2200 nM, including any value or range between these values. In some embodiments, the antibody-peptide fusion protein binds to human amyloid fibrils with a Kd that is about 40-50 nM. In some embodiments, the antibody-peptide fusion protein binds to human amyloid fibrils with a Kd that is 40-50 nM. In some embodiments, the antibody-peptide fusion protein binds to human amyloid fibrils with a Kd that is less than 50 nM. In some embodiments, the antibody-peptide fusion protein binds to human amyloid fibrils with a Kd that is less than the Kd of c11-1F4 binding to human amyloid fibrils.

In some embodiments, the antibody-peptide fusion protein binds to human amyloid fibrils with half-maximal binding at a concentration of antibody (EC₅₀) that is less than about 0.01, 0.1, or 1 μM. In some embodiments, the antibody-peptide fusion protein binds to human amyloid fibrils with half-maximal binding at a concentration of antibody (EC₅₀) that is about 0.001, 0.01, 0.05, 0.1, 0.2, 0.3, 0.4, 0.5, 0.6, 0.7, 0.8, 0.9, 1, 2, 3, 4, 5, 6, 7, 8, 9, or 10 M, including any value or range between these values. In some embodiments, the antibody-peptide fusion protein binds to human amyloid fibrils with half-maximal binding at a concentration of antibody (EC₅₀) that is less than about 1, 10, 100, or 1000 nM. In some embodiments, the antibody-peptide fusion protein binds to human amyloid fibrils with half-maximal binding at a concentration of antibody (EC₅₀) that is about 1, 5, 10, 15, 20, 25, 30, 35, 40, 45, 50, 55, 60, 65, 70, 75, 80, 85, 90, 95, 100, 100, 250, 500, 750, or 1000 nM, including any value or range between these values. In some embodiments, the antibody-peptide fusion protein binds to human amyloid fibrils with half-maximal binding at a concentration of antibody (EC₅₀) that is about 17 nM, 7 nM, 16 nM, 75 nM, or 95 nM. In some embodiments, the antibody-peptide fusion protein binds to human amyloid fibrils with half-maximal binding at a concentration of antibody (EC₅₀) that is less than about 10 nM, 20 nM, 80 nM, or 100 nM. In some embodiments, the antibody-peptide fusion protein binds to human amyloid fibrils with half-maximal binding at a concentration of antibody (EC₅₀) that is less than the EC₅₀ of chimeric 11-1F4 binding to human amyloid fibrils.

Methods for calculating dissociation constants and EC₅₀s are known in the art, and include, for example, surface plasmon resonance, enzyme-linked immunosorbent assays (ELISAs) and europium-linked immunosorbant assays (EuLISAs). In some embodiments, the dissociation constant is determined by measuring binding to a Len(1-22) monomer peptide, for example, using surface plasmon resonance. In some embodiments, the EC₅₀ is determined using a EuLISA. In some embodiments, the EC₅₀ is determined using a EuLISA to measure the level of binding to rVλ6WIL fibrils, ATTRwt human extract, ATTRv human extract, ALλ human extract, or ALκ human extract.

In some embodiments, the antibody-peptide fusion protein binds to rVλ6WIL fibrils, tATTRwt human extract, ATTRv human extract, ALλ human extract, and/or ALκ human extract. In some embodiments, the antibody-peptide fusion proteins described herein bind to amyloid deposits or fibrils. In some embodiments, the antibody-peptide fusion protein binds to one or more amyloidogenic peptides in amyloids. In some embodiments, amyloids bound by the antibody-peptide fusion protein comprise an amyloidogenic λ6 variable domain protein (Vλ6WIL) or an amyloidogenic immunoglobulin light chain (AL), Aβ(1-40) amyloid-like fibril or an amyloidogenic Aβ precursor protein, or serum amyloid protein A (AA). In other embodiments, the amyloids bound by the antibody-peptide fusion protein comprise amyloidogenic forms of immunoglobulin heavy chain (AH), β₂-microglobulin (Aβ₂M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALECT2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALκ4, ALλ1 other amyloidogenic peptides. The amyloidogenic peptides bound by the antibody-peptide fusion protein can be a protein, a protein fragment, or a protein domain. In some embodiments, the amyloid deposits or amyloid fibrils comprise recombinant amyloidogenic proteins. In some embodiments, the amyloids are part of the pathology of a disease.

In some embodiments, the antibody-peptide fusion protein exhibits one or more in vivo features selected from among improved biodistribution, pan amyloid reactivity, and inhibition of fibril growth to a reference antibody. In some embodiments, the antibody-peptide fusion protein exhibits improved biodistribution compared to reference antibody, wherein the antibody-peptide fusion protein is detectable in organs across the body. In some embodiments, the antibody-peptide fusion protein exhibits improved biodistribution, wherein the antibody-peptide fusion protein is detectable in one or more of the liver, spleen, heart, kidney, brain, muscle, pancreas, stomach, upper intestine, lower intestine, and blood. In some embodiments, the antibody-peptide fusion protein exhibits pan amyloid reactivity compared to reference antibody, wherein the antibody-peptide fusion protein is reactive towards one or more distinct amyloid substrates in vivo. In some embodiments, the antibody-peptide fusion protein is reactive towards amyloid substrates in the liver, spleen, heart, kidney, brain, muscle, pancreas, stomach, upper intestine, lower intestine, and blood. In some embodiments, the antibody-peptide fusion protein exhibits fibril growth inhibition compared to reference antibody, wherein contacting an amyloid substrate in vivo with the antibody-peptide fusion protein results in reduced growth of amyloid fibrils. In some embodiments, contacting an amyloid substrate in vivo with the antibody-peptide fusion protein results in reduced amyloid fibril growth. In some embodiments, contacting an amyloid substrate in vivo with the antibody-peptide fusion protein results in reduced amyloid fibril growth and a reduction or a slowing in the progression of an amyloid related disease. In some embodiments, contacting an amyloid substrate in vivo with the antibody-peptide fusion protein provides therapeutic benefit for an individual having an amyloid related disorder. In some embodiments, the reference antibody is not engineered to bind amyloid substrates. In some embodiments, the reference antibody does not comprise an amyloid-reactive peptide. In some embodiments, the reference antibody is not fused to an amyloid-reactive peptide. In some embodiments, the reference antibody serves as a negative control. In some embodiments, the reference antibody is an IgG antibody. In some embodiments, the reference antibody is an IgG1, IgG2, IgG3, or IgG4 isotype. In some embodiments, the reference antibody is an IgG1 isotype.

For fusion protein production, the fusion protein expression vector may be introduced into one or more appropriate production cell lines known in the art. Introduction of the expression vector may be accomplished by co-transfection via electroporation or any other suitable transformation technology available in the art. Fusion protein producing cell lines can then be selected and expanded and antibodies purified. The purified fusion protein can then be analyzed by standard techniques such as SDS-PAGE or size exclusion chromatography (SEC).

The antibody-peptide fusion protein may be made by any technique known to those of skill in the art, including chemical synthesis or recombinant means using standard molecular biological techniques (e.g., WO2022/246433).

Antibody that Binds to Amyloid Fibrils (Amyloid-Reactive Antibody)

In some embodiments, the methods provided herein comprise reducing or preventing amyloid fibril growth by administering an amyloid-reactive antibody. Alternatively, in some embodiments, the methods provided herein comprise reducing or slowing the progression of an amyloid related disease in an individual by administering an amyloid-reactive antibody.

In some embodiments, the amyloid-reactive antibody bind to an amyloid. In some embodiments, the amyloid-reactive antibody binds to amyloid of immunoglobulin light chain (AL). In some embodiments, the amyloid-reactive antibody binds to the same amyloid type as the amyloid-reactive peptide. In some embodiments, the amyloid-reactive antibody binds to a different type of amyloid than the amyloid-reactive peptide. In some embodiments, the amyloid-reactive peptide has pan-amyloid specificity as described herein. Accordingly, without being bound by this theory, the amyloid-reactive peptide of the fusion protein described herein targets the fusion protein to any amyloid deposits and the Fc domain of the amyloid-reactive antibody triggers an immune response against any type of amyloid.

In some embodiments, the antibody comprises a heavy chain comprising a heavy chain variable region (VH) and a light chain comprising a light chain variable region (VL).

In some embodiments, the amyloid-reactive antibody comprises a humanized antibody comprising the amino acid sequence of a VL as shown in Table 3. In some embodiments, the amyloid-reactive antibody comprises a humanized antibody, wherein the humanized antibody comprises a VL selected from the group consisting of VL2, VL3, VL4, VL4-N33S, VL4-N33Q, VL4-N33E, VL4-N33A, VL4-N33H, VL4-G34A, or VL4-G34V, as shown in Table 3. In some embodiments, the VL comprises an amino acid sequence set forth in the group consisting of SEQ ID NOs: 32-42. In some embodiments, the amyloid-reactive antibody comprises VL CDR amino acid sequences as shown in Table 5.

In some embodiments, the amyloid-reactive antibody comprises a humanized antibody comprising the amino acid sequence of a VH as shown in Table 4. In some embodiments, the amyloid-reactive antibody comprises a humanized antibody, wherein the humanized antibody comprises a VH selected from the group consisting of VH2, VH3, VH4, VH5, VH6, VH7, VH8, VH9, VH10, VH9-D54S, VH9-D54Q, VH9-D54E, VH9-D54A, VH9-D54H, VH9-G55A, VH9-G55V, VH9-M64V, VH9-M64I, VH9-M64L, or VH9-M64A, as shown in Table 4. In some embodiments, the VH comprises an amino acid sequence set forth in the group consisting of SEQ ID NOs: 43-63. In some embodiments, the amyloid-reactive antibody comprises VH CDR amino acid sequences as shown in Table 6.

In some embodiments, the amyloid-reactive antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22.

In some embodiments, the amyloid-reactive antibody comprises a heavy chain and a light chain, wherein the heavy chain of the antibody comprises a heavy chain variable region (VH) comprising a CDR-H1, CDR-H2, and CDR-H3 of a VH comprising the amino acid sequence set forth in SEQ ID NO:55, and the light chain of the antibody comprises a light chain variable region (VL) comprising a CDR-L1, CDR-L2, and CDR-L3 of a VL comprising the amino acid sequence set forth in SEQ ID NO:36.

In some embodiments, the antibody that binds to amyloid fibrils comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO:88, and the third and fourth polypeptide comprise the amino acid sequence set forth in SEQ ID NO:91.

In some embodiments, the antibody that binds to amyloid fibrils comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO:88, and the third and fourth polypeptide comprise the amino acid sequence set forth in SEQ ID NO:93.

### EXAMPLES

The following examples are included for illustrative purposes only and are not intended to limit the scope of the present disclosure.

### Example 1: Method for rVλ6WIL Fibril Extension with Biotinylated rVλ6WIL Monomer Mixed with an Amyloid-Reactive Peptide, an Amyloid-Reactive Antibody, or an Amyloid-Reactive Antibody-Peptide Fusion Protein

This example describes a method to analyze the elongation of rVλ6WIL (WIL) fibrils in the presence of amyloid-reactive peptides, amyloid-reactive immunoglobulins, and amyloid-reactive antibody-peptide fusion proteins.

A WIL fibril extension assay was developed for analyzing in vitro amyloid fibril formation using biotinylated WIL monomers.

First, a high binding 96-well ELISA plate was coated with 0.83 μM sonicated rVλ6WIL synthetic fibrils (50 mL per well). The fibrils were then dried in a 37° C. oven. Subsequently, the plate was washed twice with wash buffer (PBS+0.05% tween 20). The plate was then blocked with 200 mL/well of 1% BSA in PBS for 1 hour at 37° C. The blocking buffer was then removed and the plate was washed twice with wash buffer. For positive control wells, 100 mL of 20 nM biotinylated rVλ6WIL monomer (biotinylated with EZ-link Sulfo-NHS-biotin (ThermoFisher Scientific) following their protocol) in 1% BSA+0.05% tween 20 in PBS (BSAT) was added per well. For test wells, 100 mL of a mix of 20 nM biotinylated rVλ6WIL monomer plus an amyloid-reactive peptide, an amyloid-reactive antibody, or an amyloid-reactive antibody-peptide fusion protein (2, 0.5, 0.05, 0.01, 0.005 and 0.001 mM) in BSAT was added per well. Background control wells (containing no fibrils) contained 100 mL of 20 nM biotinylated rVλ6WIL monomer in BSAT added to them. The microplate was then incubated for 1 hour at 37° C. The plate was then washed twice with wash buffer. 100 mL of a 1:1000 dilution of europium-labeled streptavidin (Perkin Elmer) in BSAT was then added into each well and incubated for 1 hour at 37° C. Next, the plate was washed 3× with wash buffer. To quantify amyloid fibril formation, 100 mL of enhancement solution (Perkin Elmer) was added to each well and time-resolved fluorescence emission was measured in each well.

### Example 2: RVλ6WIL Fibril Extension in the Presence of an Amyloid-Reactive Peptide, an Amyloid-Reactive Antibody-Peptide Fusion Protein, or an Amyloid-Reactive Antibody

This example describes the level of rVλ6WIL fibril extension in the presence of an amyloid-reactive peptide p5R, an amyloid-reactive antibody-peptide fusion protein, or an amyloid-reactive antibody.

The methods of Example 1 were carried out in the presence of the amyloid reactive peptide p5R, an amyloid-reactive antibody-peptide fusion protein, an amyloid-reactive humanized antibody, or a control.

The amyloid-reactive peptide used in this example comprises peptide p5R (SEQ ID NO: 2).

The antibody-peptide fusion protein used in this example comprised an amyloid-reactive peptide comprising the amino acid sequence set forth in SEQ ID NO:2, and an antibody that binds to human amyloid fibrils, wherein the antibody comprised a heavy chain and a light chain, wherein the heavy chain of the antibody comprised a heavy chain variable region (VH) comprising the amino acid sequence set forth in SEQ ID NO: 55 and the light chain of the antibody comprised a light chain variable region (VL) comprising the amino acid sequence set forth in SEQ ID NO: 36, wherein the VH comprised a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprised a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22, wherein the amyloid-reactive peptide and antibody were linked at the C-terminal end of the light chain, and wherein the amyloid-reactive peptide was linked to the antibody via a spacer comprising an amino acid sequence set forth in SEQ ID NO:83.

The amyloid-reactive humanized antibody used in this example comprised an antibody that binds to human amyloid fibrils, wherein the antibody comprised a heavy chain and a light chain, wherein the heavy chain of the antibody comprised a heavy chain variable region (VH) comprising an amino acid sequence set forth in SEQ ID NO: 55 and the light chain of the antibody comprised a light chain variable region (VL) comprising an amino acid sequence set forth in SEQ ID NO: 36, wherein the VH comprised a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprised a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22

Assay conditions included the following: 20 nM biotinylated WIL monomer, 1 hour incubation using BSA as a blocking agent, and 50 μL/well of 10 μg/mL fibrils (0.83 μg).

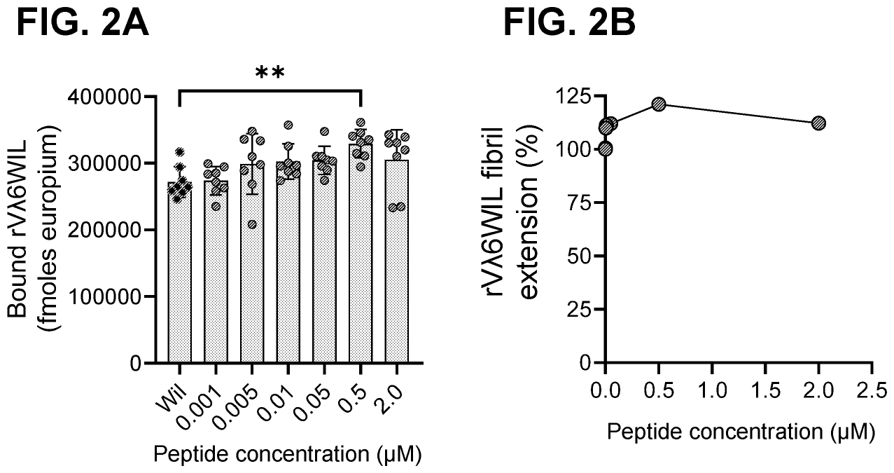

As shown in FIG. 2A and FIG. 2B, rVλ6WIL fibrils continued to extended in the presence of the amyloid-reactive peptide p5R (SEQ ID NO: 2). These results suggest that an amyloid-reactive peptide alone is insufficient to inhibit rVλ6WIL fibril extension at concentrations between 0.001 μM to 2.0 μM (FIGS. 2A, 2B).

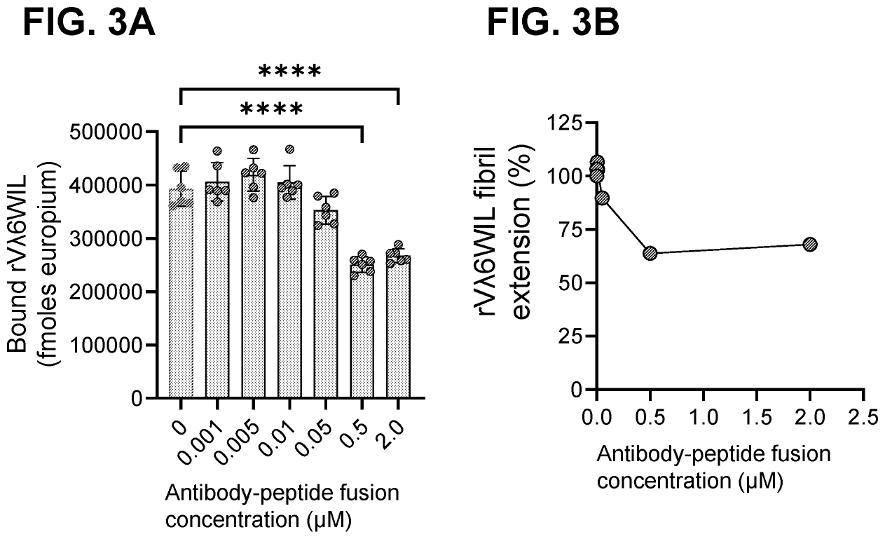

As shown in FIG. 3A and FIG. 3B, the extension of rVλ6WIL fibrils were significantly attenuated in the presence of the amyloid-reactive antibody-peptide fusion protein at a concentration of 0.5 μM or 2.0 μM. The percentage of rVλ6WIL fibril extension was similar between 0.5 μM or 2.0 μM antibody-peptide fusion protein (FIGS. 3A, 3B).

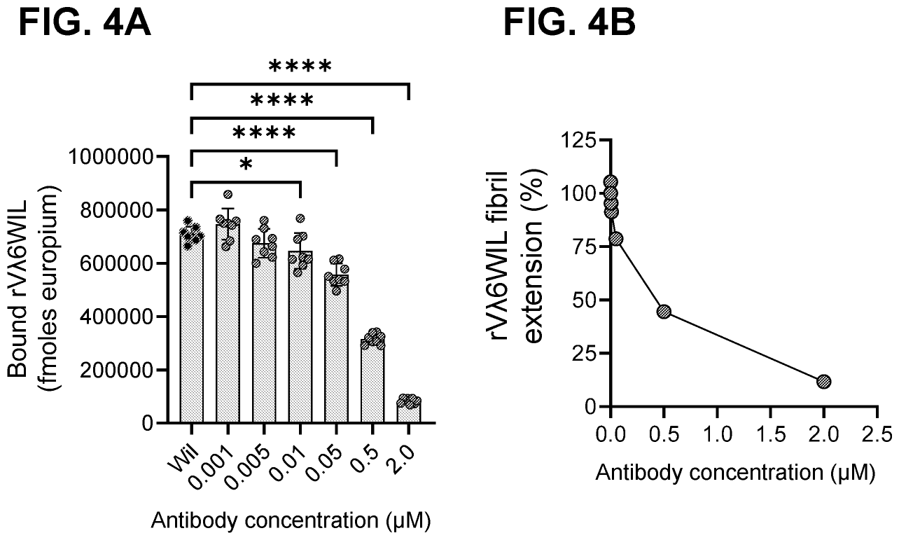

As shown in FIG. 4A and FIG. 4B, the extension of rVλ6WIL fibrils were significantly attenuated in the presence of the amyloid-reactive antibody beginning at a concentration of 0.01 μM of the amyloid-reactive antibody. The percentage of rVλ6WIL fibril extension decreased as the concentration of amyloid-reactive antibody increased. The maximum level of rVλ6WW fibril extension inhibition was observed at a concentration of 2.0 μM amyloid-reactive antibody (FIGS. 4A, 4B).

Table 10 summarizes the percentage change in rVλ6WIL fibril extension in the presence of the amyloid-reactive peptide p5R, the amyloid-reactive antibody-peptide fusion protein, or the amyloid-reactive antibody.

TABLE 10
Summary of Wil fibril extension analysis

| | Concentration | Blocking | % Change |
|---|---|---|---|
| Sample | Biotin-WIL | agent | (at 2 μM) |
| Amyloid reactive peptide p5R | 20 nM | BSA | +12% |
| Antibody-peptide fusion | 20 nM | BSA | −33% |
| Amyloid binding antibody | 20 nM | BSA | −89% |

Without wishing to be bound theory, these results suggest that the antibody-peptide fusion protein inhibits the growth of existing fibrils (e.g. recruitment of additional monomer WIL) by binding to the amyloid fibrils (long axis) and blocking recruitment sites. The amyloid-reactive antibody (metabolite surrogate) sequesters misfolded WIL VL, or WIL VL amyloidogenic higher molecular weight aggregates and prevents recruitment by the fibrils, or binds recruitment sites on the fibril in higher density or that differ from those bound by the antibody-peptide fusion protein.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

### EXEMPLARY EMBODIMENTS

Embodiments disclose herein may include:

1. A method of reducing or preventing fibril growth in an individual at risk of developing an amyloid related disease, comprising administering to the individual a therapeutically effective amount of an antibody-peptide fusion protein, wherein the antibody-peptide fusion protein comprises:
(i) an amyloid-reactive peptide; and
(ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

2. A method of reducing or slowing the progression of an amyloid related disease in an individual, wherein the individual has been diagnosed with an amyloid disease, comprising administering to the individual a therapeutically effective amount of an antibody-peptide fusion protein, wherein the antibody-peptide fusion protein comprises:
(i) an amyloid-reactive peptide; and
(ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

3. The method of embodiment 1 or embodiment 2, wherein the amyloid fibrils comprise an amyloidogenic λ6 variable domain protein (Vλ6Wil) or an amyloidogenic immunoglobulin light chain (AL), Aβ(1-40) amyloid-like fibril or an amyloidogenic Aβ precursor protein, or serum amyloid protein A (AA).

4. The method of embodiment 1 or embodiment 2, wherein the amyloid fibrils comprise amyloidogenic forms of immunoglobulin heavy chain (AH), β2-microglobulin (Aβ2M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin (ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALβ4, or ALβ1.

5. The method of any one of embodiments 1-4, wherein fibril growth is measured via an in vitro rVλ6WIL (WIL) fibril extension assay.

6. The method of any one of embodiments 1-5, wherein the antibody-peptide fusion protein reduces rVλ6WIL fibril growth by at least about 20%.

7. The method of any one of embodiments 1-5, wherein the antibody-peptide fusion protein reduces the growth rate of rVλ6WIL fibril by at least about 20%.

8. The method of any one of embodiments 1-5, wherein the antibody-peptide fusion protein reduces the total amount of rVλ6WIL fibril by at least about 20%.

9. The method of embodiment 6, wherein the antibody-peptide fusion protein reduces rVλ6WIL fibril growth by about 33%.

10. The method of embodiment 7, wherein the antibody-peptide fusion protein reduces the growth rate of rVλ6WIL fibril by about 33%.

11. The method of embodiment 8, wherein the antibody-peptide fusion protein reduces the total amount of rVλ6WIL fibril by about 33%.

12. The method of any one of embodiments 1-11, wherein the amyloid related disease is systemic or localized amyloidosis.

13. The method of any one of embodiments 1-12, wherein the amyloid related disease is selected from the group consisting of AL, AH, Aβ2M, ATTRv, ATTRwt, AA, AApoAI, AApoAII, AGel, ALys, ALECT2, AFib, ACys, ACal, AMed, AIAPP, APro, AIns, APrP, or As amyloidosis.

14. The method of any one of embodiments 1-13, wherein the individual has a genetic predisposition to an amyloid related disease.

15. The method of any one of embodiments 1-14, wherein the individual has a family history of an amyloid related disease.

16. The method of any one of embodiments 2-15, wherein the individual has an early stage of an amyloid related disease.

17. The method of any one of embodiments 2-16, wherein the individual has an early stage of AL amyloidosis.

18. The method of embodiment 17, wherein the early stage of AL amyloidosis is diagnosed according to the Mayo Clinic system.

19. The method of embodiment 18, wherein the early stage of AL amyloidosis is stage 1 AL amyloidosis.

20. The method of any one of embodiments 2-16, wherein the individual has an early stage of ATTR amyloidosis.

21. The method of embodiment 20, wherein the early stage of ATTR amyloidosis comprises stage 1 ATTR amyloidosis.

22. The method of any one of embodiments 1-21, wherein the light chain of the antibody comprises a light chain constant region, and the heavy chain of the antibody comprises a heavy chain constant region.

23. The method of any one of embodiments 1-22, wherein the spacer is selected from the group consisting of SEQ ID NOs: 23-24, 27, and 83-86.

24. The method of any one of embodiments 1-23, wherein the spacer is selected from the group consisting of SEQ ID NO:83 and SEQ ID NO:86.

25. The method of any one of embodiments 1-24, wherein the amyloid-reactive peptide comprises an amino acid sequence selected from the group consisting of SEQ ID NOs: 1-13 comprising 0, 1, 2, 3, or 4 amino acid substitutions, insertions, or deletions.

26. The method of any one of embodiments 1-25, wherein the amyloid-reactive peptide comprises an amino acid sequence selected from the group consisting of SEQ ID NOs: 1-13.

27. The method of any one of embodiments 1-26, wherein the antibody-peptide fusion protein comprises two heavy chains and two light chains, and wherein each light chain is linked at the C-terminal end of the light chain to the amyloid-reactive peptide.

28. The method of anyone of embodiments 1-27, wherein the antibody is a chimeric antibody or humanized antibody.

29. The method of any one of embodiments 1-28, wherein
the light chain of the antibody comprises a light chain variable domain (VL) comprising a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22,
and the heavy chain of the antibody comprises a heavy chain variable domain (VH) comprising a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19.

30. The method of embodiment 29, wherein the VL comprises an amino acid sequence set forth in SEQ ID NO:36, and the VH comprises an amino acid sequence set forth in SEQ ID NO:55.

31. The method of any one of embodiments 1-30, wherein the antibody is a full-length antibody comprising an Fc region.

32. The method of embodiment 31, wherein the Fc region is of an IgG1 isotype.

33. The method of any one of embodiments 1-32, wherein the antibody-peptide fusion protein comprises:
(i) the amyloid-reactive peptide comprising the amino acid sequence set forth in SEQ ID NO:1 or SEQ ID NO:2; and
(ii) the antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22;
wherein the amyloid-reactive peptide and antibody are linked at the C-terminal end of the light chain, and wherein the amyloid-reactive peptide is linked to the antibody via a spacer comprising an amino acid sequence selected from the group consisting of SEQ ID NOs: 23-24, 27, 83-86.

34. The method of any one of embodiments 1-33 wherein the antibody-peptide fusion protein comprises:
(i) the amyloid-reactive peptide comprising the amino acid sequence set forth in SEQ ID NO:2; and
(ii) the antibody that binds to a human amyloid fibrils, wherein the antibody comprises a heavy chain and a light chain, wherein the heavy chain of the antibody comprises a heavy chain variable region (VH) comprising a CDR-H1, CDR-H2, and CDR-H3 of a VH comprising the amino acid sequence set forth in SEQ ID NO:55, and the light chain of the antibody comprises a light chain variable region (VL) comprising a CDR-L1, CDR-L2, and CDR-L3 of a VL comprising the amino acid sequence set forth in SEQ ID NO:36; wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83.

35. The method of any one of embodiments 1-34, wherein the antibody-peptide fusion protein comprises:
(i) the amyloid-reactive peptide comprising the amino acid sequence set forth in SEQ ID NO:2; and
(ii) the antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22; wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83.

36. The method of any one of embodiments 1-35, wherein the antibody-peptide fusion protein comprises a first polypeptide and a second polypeptide each comprising a light chain of the antibody and an amyloid-reactive peptide, and a third and a fourth polypeptide each comprising a heavy chain of the antibody, and wherein the first polypeptide and second polypeptide each comprises the amino acid set forth in SEQ ID NO:89, and the third and fourth polypeptide each comprises the amino acid sequence set forth in SEQ ID NO:91.

37. The method of any one of embodiments 1-35, wherein the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide each comprises the amino acid set forth in SEQ ID NO:89, and the third and fourth polypeptide each comprises the amino acid sequence set forth in SEQ ID NO:93.

38. The method of any one of embodiments 1-37, wherein the individual is a human.

39. A method of reducing or preventing fibril growth in an individual having an amyloid related disease, comprising administering to the individual a therapeutically effective amount of an antibody-peptide fusion protein, wherein the antibody-peptide fusion protein comprises:
(i) an amyloid-reactive peptide; and
(ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

40. A method of reducing or preventing fibril growth in an individual in whom amyloid fibrils have been identified, comprising administering to the individual a therapeutically effective amount of an antibody-peptide fusion protein, wherein the antibody-peptide fusion protein comprises:
(i) an amyloid-reactive peptide; and
(ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

41. A method of reducing or preventing fibril growth in an individual, wherein the individual has been diagnosed with an amyloid disease, comprising administering to the individual a therapeutically effective amount of an antibody-peptide fusion protein, wherein the antibody-peptide fusion protein comprises:
(i) an amyloid-reactive peptide; and
(ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

1. A method of reducing or preventing fibril growth in an individual at risk of developing an amyloid related disease, comprising administering to the individual a therapeutically effective amount of an antibody-peptide fusion protein, wherein the antibody-peptide fusion protein comprises:
(i) an amyloid-reactive peptide, wherein the amyloid-reactive peptide comprises an amino acid sequence selected from the group consisting of SEO ID NOs: 1-13; and
(ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

2. A method of reducing or slowing the progression of an amyloid related disease in an individual, wherein the individual has been diagnosed with an amyloid disease, comprising administering to the individual a therapeutically effective amount of an antibody-peptide fusion protein, wherein the antibody-peptide fusion protein comprises:
(i) an amyloid-reactive peptide, wherein the amyloid-reactive peptide comprises an amino acid sequence selected from the group consisting of SEO ID NOs: 1-13; and
(ii) an antibody that binds to amyloid fibrils, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

3. The method of claim 1, wherein the amyloid fibrils comprise an amyloidogenic λ6 variable domain protein (Vλ6Wil) or an amyloidogenic immunoglobulin light chain (AL), Aβ(1-40) amyloid-like fibril or an amyloidogenic Aβ precursor protein, or serum amyloid protein A (AA).

4. The method of claim 1, wherein the amyloid fibrils comprise amyloidogenic forms of immunoglobulin heavy chain (AH), β2-microglobulin (Aβ2M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin (ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALβ4, or ALP 1.

5. The method of claim 1, wherein fibril growth is measured via an in vitro rVλ6WIL (WIL) fibril extension assay.

6.-11. (canceled)

12. The method of claim 1, wherein the amyloid related disease is systemic amyloidosis.

13. The method of claim 1, wherein the amyloid related disease is selected from the group consisting of AL, AH, Aβ2M, ATTRv, ATTRwt, AA, AApoAI, AApoAII, AGel, ALys, ALECT2, AFib, ACys, ACal, AMed, AIAPP, APro, AIns, APrP, or Aβ amyloidosis.

14. The method of claim 1, wherein the individual has a genetic predisposition to an amyloid related disease.

15. The method of claim 1, wherein the individual has a family history of an amyloid related disease.

16.-21. (canceled)

22. The method of claim 1, wherein the light chain of the antibody comprises a light chain constant region, and the heavy chain of the antibody comprises a heavy chain constant region.

23. (canceled)

24. The method of claim 1, wherein the spacer is selected from the group consisting of SEQ ID NO:83 and SEQ ID NO:86.

25.-26. (canceled)

27. The method of claim 1, wherein the antibody-peptide fusion protein comprises two heavy chains and two light chains, and wherein each light chain is linked at the C-terminal end of the light chain to the amyloid-reactive peptide.

28. The method of claim 1, wherein the antibody is a humanized antibody.

29. The method of claim 1, wherein
the light chain of the antibody comprises a light chain variable domain (VL) comprising a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22,
and the heavy chain of the antibody comprises a heavy chain variable domain (VH) comprising a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19.

30. The method of claim 29, wherein the VL comprises an amino acid sequence set forth in SEQ ID NO:36, and the VH comprises an amino acid sequence set forth in SEQ ID NO:55.

31. The method of claim 1, wherein the antibody is a full-length antibody comprising an Fc region.

32. The method of claim 31, wherein the Fc region is of an IgG1 isotype.

33.-34. (canceled)

35. The method of claim 1, wherein the antibody-peptide fusion protein comprises:
(i) the amyloid-reactive peptide comprising the amino acid sequence set forth in SEQ ID NO:2; and
(ii) the antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:17, a CDR-H2 comprising the amino acid sequence set forth in SEQ ID NO:73, and a CDR-H3 comprising the amino acid sequence set forth in SEQ ID NO:19, and the VL comprises a CDR-L1 comprising the amino acid sequence set forth in SEQ ID NO:64, a CDR-L2 comprising the amino acid sequence set forth in SEQ ID NO:21, and a CDR-L3 comprising the amino acid sequence set forth in SEQ ID NO:22; wherein the amyloid-reactive peptide and antibody are linked at the C-terminal end of the light chain, and wherein the amyloid-reactive peptide is linked to the antibody via a spacer comprising an amino acid sequence set forth in SEQ ID NO:83.

36. The method of claim 1, wherein the antibody-peptide fusion protein comprises a first polypeptide and a second polypeptide each comprising a light chain of the antibody and an amyloid-reactive peptide, and a third and a fourth polypeptide each comprising a heavy chain of the antibody, and wherein the first polypeptide and second polypeptide each comprises the amino acid set forth in SEQ ID NO:89, and the third and fourth polypeptide each comprises the amino acid sequence set forth in SEQ ID NO:91.

37. The method of claim 1, wherein the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide each comprises the amino acid set forth in SEQ ID NO:89, and the third and fourth polypeptide each comprises the amino acid sequence set forth in SEQ ID NO:93.

38. (canceled)


SEQUENCE LISTING (93 sequences)

SEQ ID NO: 1 - protein, 26 aa, synthetic construct
KAQKAQAKQAKQAQKAQKAQAKQAKQ

SEQ ID NO: 2 - protein, 26 aa, synthetic construct
RAQRAQARQARQAQRAQRAQARQARQ

SEQ ID NO: 3 - protein, 15 aa, synthetic construct
KAKAKAKAKAKAKAK

SEQ ID NO: 4 - protein, 29 aa, synthetic construct
KAQAKAQAKAQAKAQAKAQAKAQAKAQAK

SEQ ID NO: 5 - protein, 26 aa, synthetic construct
KAQQAQAKQAQQAQKAQQAQAKQAQQ

SEQ ID NO: 6 - protein, 26 aa, synthetic construct
QAQKAQAQQAKQAQQAQKAQAQQAKQ

SEQ ID NO: 7 - protein, 26 aa, synthetic construct
KAQKAQAKQAKQAQKAQKAQAKQAKQ

SEQ ID NO: 8 - protein, 26 aa, synthetic construct
KTVKTVTKVTKVTVKTVKTVTKVTKV

SEQ ID NO: 9 - protein, 18 aa, synthetic construct
VYKVKTKVKTKVKTKVKT

SEQ ID NO: 10 - protein, 30 aa, synthetic construct
AQAYSKAQKAQAKQAKQAQKAQKAQAKAKQ

SEQ ID NO: 11 - protein, 31 aa, synthetic construct
AQAYARAQRAQARQARQAQRAQRAQARQARQ

SEQ ID NO: 12 - protein, 40 aa, synthetic construct
KAQKAQAKQAKQAQKAQKAQAKQAKQAQKAQKAQAKQAKQ

SEQ ID NO: 13 - protein, 40 aa, synthetic construct
RAQRAQARQARQAQRAQRAQARQARQAQRAQRAQARQARQ

SEQ ID NO: 14 - skipped

SEQ ID NO: 15 - protein, 111 aa, synthetic construct
QVQLKESGPGLVAPSQSLSITCTVSGFSLSSYGVSWVRQPPGKGLEWLGVIWGDGSTNYHPNLMSRLSISKDISKSQVLFKLNSLQTDDTATYYCVTLDYWGQGTSVTVSS

SEQ ID NO: 16 - protein, 112 aa, synthetic construct
DVVMTQTPLSLPVSLGDQASISCRSSQSLVHRNGNTYLHWYLQKPGQSPKLLIYKVSNRFSGVPDRFSGSGSGTDFTLKISRVEAEDLGLYFCFQTTYVPNTFGGGTKLEIK

SEQ ID NO: 17 - protein, 10 aa, synthetic construct
GFSLSSYGVS

SEQ ID NO: 18 - protein, 16 aa, synthetic construct
VIWGDGSTNYHPNLMS

SEQ ID NO: 19 - skipped

SEQ ID NO: 20 - protein, 16 aa, synthetic construct
RSSQSLVHRNGNTYLH

SEQ ID NO: 21 - protein, 7 aa, synthetic construct
KVSNRFS

SEQ ID NO: 22 - protein, 9 aa, synthetic construct
FQTTYVPNT

SEQ ID NO: 23 - protein, 15 aa, synthetic construct
AQAGQAGQAQGGGYS

SEQ ID NO: 24 - protein, 5 aa, synthetic construct
VTPTV

SEQ ID NO: 25-26 - skipped (2 entries)

SEQ ID NO: 27 - protein, 5 aa, synthetic construct
GGGYS

SEQ ID NO: 28-31 - skipped (4 entries)

SEQ ID NO: 32 - protein, 112 aa, synthetic construct
DVVMTQSPLSLPVTLGQPASISCRSSQSLVHRNGNTYLHWFQQRPGQSPRRLIYKVSNRFSGVPDRFSGSGSGTDFTLKISRVEAEDVGVYYCFQTTYVPNTFGGGTKLEIK

SEQ ID NO: 33 - protein, 112 aa, synthetic construct
DVVMTQSPLSLPVTLGQPASISCRSSQSLVHRNGNTYLHWYLQRPGQSPRRLIYKVSNRFSGVPDRFSGSGSGTDFTLKISRVEAEDVGVYYCFQTTYVPNTFGGGTKLEIK

SEQ ID NO: 34 - protein, 112 aa, synthetic construct
DVVMTQSPLSLPVTLGQPASISCRSSQSLVHRNGNTYLHWYLQRPGQSPRLLIYKVSNRFSGVPDRFSGSGSGTDFTLKISRVEAEDVGLYFCFQTTYVPNTFGGGTKLEIK

SEQ ID NO: 35 - protein, 112 aa, synthetic construct
DVVMTQSPLSLPVTLGQPASISCRSSQSLVHRNGNTYLHWFQQRPGQSPRLLIYKVSNRFSGVPDRFSGSGSGTDFTLKISRVEAEDVGVYFCFQTTYVPNTFGGGTKLEIK

SEQ ID NO: 36 - protein, 112 aa, synthetic construct
DVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIK

SEQ ID NO: 37 - protein, 112 aa, synthetic construct
DVVMTQSPLSLPVTLGQPASISCRSSQSLVHRQGNTYLHWFQQRPGQSPRLLIYKVSNRFSGVPDRFSGSGSGTDFTLKISRVEAEDVGVYFCFQTTYVPNTFGGGTKLEIK

SEQ ID NO: 38 - protein, 112 aa, synthetic construct
DVVMTQSPLSLPVTLGQPASISCRSSQSLVHREGNTYLHWFQQRPGQSPRLLIYKVSNRFSGVPDRFSGSGSGTDFTLKISRVEAEDVGVYFCFQTTYVPNTFGGGTKLEIK

SEQ ID NO: 39 - protein, 112 aa, synthetic construct
DVVMTQSPLSLPVTLGQPASISCRSSQSLVHRAGNTYLHWFQQRPGQSPRLLIYKVSNRFSGVPDRFSGSGSGTDFTLKISRVEAEDVGVYFCFQTTYVPNTFGGGTKLEIK

SEQ ID NO: 40 - protein, 112 aa, synthetic construct
DVVMTQSPLSLPVTLGQPASISCRSSQSLVHRHGNTYLHWFQQRPGQSPRLLIYKVSNRFSGVPDRFSGSGSGTDFTLKISRVEAEDVGVYFCFQTTYVPNTFGGGTKLEIK

SEQ ID NO: 41 - protein, 112 aa, synthetic construct; identical to SEQ ID NO: 39

SEQ ID NO: 42 - protein, 112 aa, synthetic construct
DVVMTQSPLSLPVTLGQPASISCRSSQSLVHRVGNTYLHWFQQRPGQSPRLLIYKVSNRFSGVPDRFSGSGSGTDFTLKISRVEAEDVGVYFCFQTTYVPNTFGGGTKLEIK

SEQ ID NO: 43 - protein, 111 aa, synthetic construct
QVQLQESGPGLVKPSETLSLTCTVSGFSLSSYGVSWIRQPPGKGLEWIGVIWGDGSTNYHPNLMSRVTISVDTSKNQFSLKLSSVTAADTAVYYCARLDYWGQGTSVTVSS

SEQ ID NO: 44 - protein, 111 aa, synthetic construct
QVQLQESGPGLVKPSETLSLTCTVSGFSLSSYGVSWVRQPPGKGLEWLGVIWGDGSTNYHPNLMSRVTISVDTSKNQFSLKLSSVTAADTAVYYCARLDYWGQGTSVTVSS

SEQ ID NO: 45 - protein, 111 aa, synthetic construct
QVQLQESGPGLVKPSETLSLTCTVSGFSLSSYGVSWIRQPPGKGLEWIGVIWGDGSTNYHPNLMSRLSISVDTSKNQFSLKLSSVTAADTATYYCVTLDYWGQGTSVTVSS

SEQ ID NO: 46 - protein, 111 aa, synthetic construct
QVQLQESGPGLVKPSETLSLTCTVSGFSLSSYGVSWVRQPPGKGLEWLGVIWGDGSTNYHPNLMSRLSISVDTSKNQFSLKLSSVTAADTAVYYCARLDYWGQGTSVTVSS

SEQ ID NO: 47 - protein, 111 aa, synthetic construct
QVQLQESGPGLVKPSETLSLTCTVSGFSLSSYGVSWVRQPPGKGLEWLGVIWGDGSTNYHPNLMSRLSISVDTSKNQFSLKLSSVTAADTAVYYCVTLDYWGQGTSVTVSS

SEQ ID NO: 48 - protein, 111 aa, synthetic construct
QVQLQESGPGLVKPSETLSLTCTVSGFSLSSYGVSWVRQPPGKGLEWLGVIWGDGSTNYHPNLMSRLSISKDTSKNQFSLKLSSVTAADTATYYCVTLDYWGQGTSVTVSS

SEQ ID NO: 49 - protein, 111 aa, synthetic construct
QVQLQESGPGLVKPSETLSLTCTVSGFSLSSYGVSWIRQPPGKGLEWIGVIWGDGSTNYHPNLMSRVTISKDTSKNQVLLKLSSVTAADTAVYYCVTLDYWGQGTSVTVSS

SEQ ID NO: 50 - protein, 111 aa, synthetic construct
QVQLQESGPGLVKPSETLSLTCTVSGFSLSSYGVSWIRQPPGKGLEWIGVIWGDGSTNYHPNLMSRVTISKDTSKSQFSLKLSSVTAADTAVYYCVTLDYWGQGTSVTVSS

SEQ ID NO: 51 - protein, 111 aa, synthetic construct
QVQLQESGPGLVKPSETLSLTCTVSGFSLSSYGVSWIRQPPGKGLEWLGVIWGDGSTNYHPNLMSRVTISVDTSKSQVLFKLSSVTAADTAVYYCATLDYWGQGTSVTVSS

SEQ ID NO: 52 - protein, 111 aa, synthetic construct
QVQLQESGPGLVKPSETLSLTCTVSGFSLSSYGVSWIRQPPGKGLEWLGVIWGDGSTNYHPNLMSRLSISKDTSKSQVLLKLSSVTAADTAVYYCVTLDYWGQGTSVTVSS

SEQ ID NO: 53 - protein, 111 aa, synthetic construct
QVQLQESGPGLVKPSETLSLTCTVSGFSLSSYGVSWIRQPPGKGLEWLGVIWGSGSTNYHPNLMSRVTISVDTSKSQVLFKLSSVTAADTAVYYCATLDYWGQGTSVTVSS

SEQ ID NO: 54 - protein, 111 aa, synthetic construct
QVQLQESGPGLVKPSETLSLTCTVSGFSLSSYGVSWIRQPPGKGLEWLGVIWGQGSTNYHPNLMSRVTISVDTSKSQVLFKLSSVTAADTAVYYCATLDYWGQGTSVTVSS

SEQ ID NO: 55 - protein, 111 aa, synthetic construct
QVQLQESGPGLVKPSETLSLTCTVSGFSLSSYGVSWIRQPPGKGLEWLGVIWGEGSTNYHPNLMSRVTISVDTSKSQVLFKLSSVTAADTAVYYCATLDYWGQGTSVTVSS

SEQ ID NO: 56 - protein, 111 aa, synthetic construct
QVQLQESGPGLVKPSETLSLTCTVSGFSLSSYGVSWIRQPPGKGLEWLGVIWGAGSTNYHPNLMSRVTISVDTSKSQVLFKLSSVTAADTAVYYCATLDYWGQGTSVTVSS

SEQ ID NO: 57 - protein, 111 aa, synthetic construct
QVQLQESGPGLVKPSETLSLTCTVSGFSLSSYGVSWIRQPPGKGLEWLGVIWGHGSTNYHPNLMSRVTISVDTSKSQVLFKLSSVTAADTAVYYCATLDYWGQGTSVTVSS

SEQ ID NO: 58 - protein, 111 aa, synthetic construct
QVQLQESGPGLVKPSETLSLTCTVSGFSLSSYGVSWIRQPPGKGLEWLGVIWGDASTNYHPNLMSRVTISVDTSKSQVLFKLSSVTAADTAVYYCATLDYWGQGTSVTVSS

SEQ ID NO: 59 - protein, 111 aa, synthetic construct
QVQLQESGPGLVKPSETLSLTCTVSGFSLSSYGVSWIRQPPGKGLEWLGVIWGDVSTNYHPNLMSRVTISVDTSKSQVLFKLSSVTAADTAVYYCATLDYWGQGTSVTVSS

SEQ ID NO: 60 - protein, 111 aa, synthetic construct
QVQLQESGPGLVKPSETLSLTCTVSGFSLSSYGVSWIRQPPGKGLEWLGVIWGDGSTNYHPNLVSRVTISVDTSKSQVLFKLSSVTAADTAVYYCATLDYWGQGTSVTVSS

SEQ ID NO: 61 - protein, 111 aa, synthetic construct
QVQLQESGPGLVKPSETLSLTCTVSGFSLSSYGVSWIRQPPGKGLEWLGVIWGDGSTNYHPNLISRVTISVDTSKSQVLFKLSSVTAADTAVYYCATLDYWGQGTSVTVSS

SEQ ID NO: 62 - protein, 111 aa, synthetic construct
QVQLQESGPGLVKPSETLSLTCTVSGFSLSSYGVSWIRQPPGKGLEWLGVIWGDGSTNYHPNLLSRVTISVDTSKSQVLFKLSSVTAADTAVYYCATLDYWGQGTSVTVSS

SEQ ID NO: 63 - protein, 111 aa, synthetic construct
QVQLQESGPGLVKPSETLSLTCTVSGFSLSSYGVSWIRQPPGKGLEWLGVIWGDGSTNYHPNLASRVTISVDTSKSQVLFKLSSVTAADTAVYYCATLDYWGQGTSVTVSS

SEQ ID NO: 64 - protein, 16 aa, synthetic construct
RSSQSLVHRSGNTYLH

SEQ ID NO: 65 - protein, 16 aa, synthetic construct
RSSQSLVHRQGNTYLH

SEQ ID NO: 66 - protein, 16 aa, synthetic construct
RSSQSLVHREGNTYLH

SEQ ID NO: 67 - protein, 16 aa, synthetic construct
RSSQSLVHRAGNTYLH

SEQ ID NO: 68 - protein, 16 aa, synthetic construct
RSSQSLVHRHGNTYLH

SEQ ID NO: 69 - protein, 16 aa, synthetic construct
RSSQSLVHRNANTYLH

SEQ ID NO: 70 - protein, 16 aa, synthetic construct
RSSQSLVHRNVNTYLH

SEQ ID NO: 71 - protein, 16 aa, synthetic construct
VIWGSGSTNYHPNLMS

SEQ ID NO: 72 - protein, 16 aa, synthetic construct
VIWGQGSTNYHPNLMS

SEQ ID NO: 73 - protein, 16 aa, synthetic construct
VIWGEGSTNYHPNLMS

SEQ ID NO: 74 - protein, 16 aa, synthetic construct
VIWGAGSTNYHPNLMS

SEQ ID NO: 75 - protein, 16 aa, synthetic construct
VIWGHGSTNYHPNLMS

SEQ ID NO: 76 - protein, 16 aa, synthetic construct
VIWGDASTNYHPNLMS

SEQ ID NO: 77 - protein, 16 aa, synthetic construct
VIWGDVSTNYHPNLMS

SEQ ID NO: 78 - protein, 16 aa, synthetic construct
VIWGDGSTNYHPNLVS

SEQ ID NO: 79 - protein, 16 aa, synthetic construct
VIWGDGSTNYHPNLIS

SEQ ID NO: 80 - protein, 16 aa, synthetic construct
VIWGDGSTNYHPNLLS

SEQ ID NO: 81 - protein, 16 aa, synthetic construct
VIWGDGSTNYHPNLAS

SEQ ID NO: 82 - skipped

SEQ ID NO: 83 - protein, 5 aa, synthetic construct
VSPSV

SEQ ID NO: 84 - protein, 10 aa, synthetic construct
VSPSVVSPSV

SEQ ID NO: 85 - protein, 5 aa, synthetic construct
GGSGG

SEQ ID NO: 86 - protein, 10 aa, synthetic construct
GGGGSGGGGS

SEQ ID NO: 87 - protein, 255 aa, synthetic construct
APGGGRAQRAQARQARQAQRAQRAQARQARQVSPSVDVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 219 aa, synthetic construct
DVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 250 aa, synthetic construct
DVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECVSPSVRAQRAQARQARQAQRAQRAQARQARQ

SEQ ID NO: 90 - protein, 255 aa, synthetic construct
DVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRAQRAQARQARQAQRAQRAQARQARQ

SEQ ID NO: 91 - protein, 441 aa, synthetic construct
QVQLQESGPGLVKPSETLSLTCTVSGFSLSSYGVSWIRQPPGKGLEWLGVIWGEGSTNYHPNLMSRVTISVDTSKSQVLFKLSSVTAADTAVYYCATLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 472 aa, synthetic construct
QVQLQESGPGLVKPSETLSLTCTVSGFSLSSYGVSWIRQPPGKGLEWLGVIWGEGSTNYHPNLMSRVTISVDTSKSQVLFKLSSVTAADTAVYYCATLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVSPSVRAQRAQARQARQAQRAQRAQARQARQ

SEQ ID NO: 93 - protein, 440 aa, synthetic construct
QVQLQESGPGLVKPSETLSLTCTVSGFSLSSYGVSWIRQPPGKGLEWLGVIWGEGSTNYHPNLMSRVTISVDTSKSQVLFKLSSVTAADTAVYYCATLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG